SEQUENCE LISTING (9 sequences)

SEQ ID NO: 1 - RNA, 23 nt, Homo sapiens
ugugcaaaucuaugcaaaacuga

SEQ ID NO: 2 - RNA, 22 nt, Homo sapiens
uuaucagaaucuccagggguac

SEQ ID NO: 3 - RNA, 24 nt, Homo sapiens
acaaaaaaaaaagcccaacccuuc

SEQ ID NO: 4 - RNA, 22 nt, Homo sapiens
ucucccaacccuuguaccagug

SEQ ID NO: 5 - RNA, 22 nt, Homo sapiens
ucguaccgugaguaauaaugcg

SEQ ID NO: 6 - RNA, 21 nt, Homo sapiens
uuaagacuugcagugauguuu

SEQ ID NO: 7 - DNA, 24 bp, Artificial
  Synthetic CCD1A-M1
gtgcgaggccagaggccacttctg

SEQ ID NO: 8 - DNA, 24 bp, Artificial
  Synthetic CCD1A-M2
gaccctgagagagctcctgagagc

SEQ ID NO: 9 - DNA, 24 bp, Artificial
  Synthetic CCD1A-M3
ttcccacctcttctggcccagagg

(19) United States
(12) Patent Application Publication
(10) Pub. No.: US 2023/0295728 A1
(43) Pub. Date: Sep. 21, 2023

(54) METHODS FOR DIAGNOSING AND TREATING AUTISM

(71) Applicants: CENTRE NATIONAL DE LA RECHERCHE SCIENTIFIQUE (CNRS), Paris (FR); INSERM (INSTITUT NATIONAL DE LA SANTÉ ET DE LA RECHERCH MÉDICALE, Paris (FR); UNIVERSITE COTE D'AZUR, Nice (FR); ERCIYES UNIVERSITY - FACULTY OF MEDICINE, Kayseri (TR)

(72) Inventors: Minoo RASSOULZADEGAN, Nice (FR); Elif Funda SENER, Kayseri (TR); Yusuf OZKUL, Kayseri (TR); Serpil TAHERI, Kayseri (TR)

(21) Appl. No.: 18/000,628

(22) PCT Filed: Jun. 3, 2021
(86) PCT No.: PCT/EP2021/064952

§ 371 Date: Dec. 2, 2022

(30) Foreign Application Priority Data
Jun. 4, 2020 (EP) 20305592.6

(51) Int. Cl. C12Q 1/6883; A61K 45/06

(52) U.S. Cl. CPC C12Q 1/6883; A61K 45/06; C12Q 2600/158; C12Q 2600/178

(57) ABSTRACT

The present invention relates generally to a method for diagnosing autism spectrum disorder (ASD) in a biological sample obtained from a subject comprising the steps of: i) extracting the miRNAs from the biological sample, ii) determining the level of at least one miRNA selected from miR-19a-3p, miR-361-5p, miR-3613-3p, miR-150-5p, miR-126-3p, and/or miR-499a-5p in the nucleic acid extraction; iii) comparing the level measured at step ii) with its predetermined reference value, and iv) concluding that the subject suffers from ASD when the level of at least one miR-19a-3p, miR-361-5p, miR-3613-3p, miR-150-5p, miR-126-3p, and/or miR-499a-5p is lower than its predetermined reference value or concluding that the subject does not suffer from ASD when the level of at least one miR-19a-3p, miR-361-5p, miR-3613-3p, miR-150-5p, miR-126-3p, and/or miR-499a-5p is the same value as its predetermined reference value. Inventors report a characteristic miRNA profile of expression of six miRNA genes detected by quantitative qRT-PCR analysis in human patients and further evidenced in two established animal models of the disease. MicroRNAs miR-19a-3p, miR-361-5p, miR-3613-3p, miR-150-5p, miR-126-3p, and miR-499a-5p were found to be expressed at low to very low levels in the serum of 45 human patients with autism.

### FIELD OF THE INVENTION

The present invention relates generally to the field of neurology. More specifically, the present invention relates to methods and kits for predicting autism.

### BACKGROUND OF THE INVENTION

Autism spectrum disorders (ASDs) encompass a range of disorders characterized by impaired social interactions and communications, together with repetitive stereotypic behaviors (references 1-5 for recent reviews). The genetic architecture underlying the range of ASD symptoms has been investigated (reviewed by Iakoucheva et al¹). Mutations in more than 100 genes involved in brain development and neural activity have been identified in patients and are thought to confer a risk for ASD^{2,3}, but a constant association that would suggest a causal relationship has not been observed. The same conclusion was recently reached from a large-scale exon sequencing analysis⁴. Hence, mouse models that reproduce characteristic elements of the disease have been developed⁵. As in other instances, attention has recently been focused on a peculiar class of regulatory alterations that modifies noncoding (nc) RNAs⁶ with putative regulatory functions in the synthesis of proteins. One class of these alterations comprises the genes encoding 22 nt-long RNA (often abbreviated miRNAs) that regulate the expression of homologous target genes by blocking translation and inducing the degradation of the mRNAs⁷. Among the miRNA genes in the mammalian genome (several hundred in the human genome), a large subset is expressed in the brain⁸, and dysfunctions of particular miRNAs have been tentatively associated with neuropathological conditions, including ASD⁹⁻¹⁰, with however diverging patterns of expression. They reflect still unknown complexities of the disease itself but they may also be the result of different analytical protocols.

Accordingly, there is a need to study miRNA and find new tools to predict autism and thus allow a best patient management.

### SUMMARY OF THE INVENTION

The present invention relates a method for diagnosing autism spectrum disorder (ASD) in a biological sample obtained from a subject comprising the steps of i) extracting the miRNAs from the biological sample, ii) determining the level of at least miR-19a-3p, miR-126-3p, miR-499a-5p, miR-361-5p, miR-3613-3p and/or miR-150-5p, in the nucleic acid extraction; iii) comparing the level measured at step ii) with its predetermined reference value, and iv) concluding that the subject suffers from ASD when the level of at least one miR-19a-3p, miR-126-3p, miR-499a-5p, miR-361-5p, miR-3613-3p and/or miR-150-5p is lower than its predetermined reference value (i.e. controls, especially healthy genitors and siblings) or concluding that the subject does not suffer from ASD when the level of at least one miR-19a-3p, miR-126-3p, miR-499a-5p, miR-361-5p, miR-3613-3p, and/or miR-150-5p is the same value as its predetermined reference control value.

In particular, the invention is defined by claims.

### DETAILED DESCRIPTION OF THE INVENTION

Here, inventors report a characteristic miRNA profile of expression of six miRNA genes detected by quantitative qRT-PCR analysis in human patients and further evidenced in two established animal models of the disease. MicroRNAs miR-19a-3p, miR-361-5p, miR-3613-3p, miR-150-5p, miR-126-3p, and miR-499a-5p were found to be expressed at low to very low levels in the serum of 45 human patients with autism. The clinically healthy progenitors and siblings of the patients showed levels of these microRNAs intermediate between those of controls and the reduced expression of patients. The same pattern was observed in two mouse models, one generated via the injection of valproic acid (VPA), a drug known to induce autism in humans^{11,12,13} and rodents^{14,15} and another one resulting from heterozygosity of the Cc2d1a^{+/-} locus, a gene of the ASD constellation encoding a transcriptional repressor of serotonin receptors^{16,17}. Among more than one-hundred gene reported to exhibit an altered expression pattern in human ASD instances, mice with a Cc2d1a mutation were chosen because, the loss of the gene affects serotonin receptors involved in the normal and pathological brain development^{18,19,20} and mutants were considered as valuable models of ASD. The same abnormal, disease-associated profile of expression of the same six microRNAs genes, in 45 patients, from multiplex (more than one child with autism) and simplex (one child with autism) families compared with their families and controls further extended to two of the established animal models.

### Method for Diagnosing Autism Spectrum Disorder (ASD)

Accordingly, in a first aspect, the invention relates to a method for diagnosing autism spectrum disorder (ASD) in a biological sample obtained from a subject comprising the steps of i) extracting the miRNAs from the biological sample, ii) determining the level of at least one miR-19a-3p, miR-126-3p, miR-499a-5p, miR-361-5p, miR-3613-3p and/or miR-150-5p, in the nucleic acid extraction; iii) comparing the level measured at step ii) with its predetermined reference value, and iv) concluding that the subject suffers from ASD when the level of at least one miR-19a-3p, miR-126-3p, miR-499a-5p, miR-361-5p, miR-3613-3p and/or miR-150-5p is lower than its predetermined reference value (i.e. controls, especially healthy genitors and siblings) or concluding that the subject does not suffer from ASD when the level of at least one miR-19a-3p, miR-126-3p, miR-499a-5p, miR-361-5p, miR-3613-3p and/or miR-150-5p is the same value as its predetermined reference value.

As used herein term “diagnosing” refers to classifying a disease or a symptom, determining a severity of the disease, monitoring disease progression, forecasting an outcome of a disease and/or prospects of recovery.

As used herein, the term “autism spectrum disorder” (ASD) refers to complex neuro developmental disabilities characterized by stereotypic behaviors and deficits in communication and social interaction. The term “spectrum” refers to the wide range of symptoms, skills, and levels of impairment, or disability, that patients with ASD can have. ASD is generally diagnosed according to guidelines listed in the Diagnostic and Statistical Manual of Mental Disorders, Fith Edition (DSM-V). The manual currently defines five disorders, sometimes called pervasive developmental disorders (PDDs), as ASD, including Autistic disorder (classic autism), Asperger's disorder (Asperger syndrome), Pervasive developmental disorder not otherwise specified (PDD-NOS), Rett's disorder (Rett syndrome), and Childhood disintegrative disorder (CDD). Some patients are mildly impaired by their symptoms, but others are severely disabled. ASD encompasses a set of complex disorders with poorly defined etiologies, and no targeted cure. Disorders include autism, Asperger syndrome, pervasive developmental disorder not otherwise specified, and childhood disintegrative disorder.

As used herein, the term “non-syndromic autism” describes cases wherein autism is the primary diagnosis and is caused by unknown genetic or environmental cause, oligogenic, polygenic, and multifactorial mechanisms. Moreover many genes involved in non-syndromic intellectual disabilities (ID) and in epilepsy have also been implicated in the etiology of nonsyndromic ASD. These genes probably belong to a continuum of neurodevelopment disorders that manifest in different manners depending on associated genetic and environmental factors.

As used herein, the term “syndromic autism” or “secondary autism” is used to refer to a condition caused by a well-known genetic variant, such as tuberous sclerosis, Rett syndrome, fragile X syndrome or other medical genetic conditions. It is typically associated with malformations and/or dysmorphic features and unlike ‘idiopathic’ ASD.

As used herein, the term “subject” refers to any mammals, such as a rodent, a feline, a canine, and a primate. Particularly, in the present invention, the subject is a human. In a further embodiment, the subject is a children. In another embodiment, the subject is a children with their relatives (parents and sibling). In a particular embodiment, the subject is a human who is susceptible to have ASD. In a particular embodiment, the subject is a human who is susceptible to have non-syndromic autism. In a particular embodiment, the subject is a human who is susceptible to have syndromic autism. Typically, said subject has or is susceptible to have stereotypic behaviors and deficits in communication and social interaction. In the context of the invention, parents and sibling of the ASD patients are suspected to have intermediary level of these six-miRNAs (miR-19a-3p, miR-361-5p, miR-3613-3p, miR-150-5p, miR-126-3p, and miR-499a-5p) compared to children with ASD and controls.

As used herein, the term “biological sample” refers to a sample obtained from a subject, for example blood, saliva, breast milk, urine, semen, blood plasma, synovial fluid, hippocampus or serum.

In a particular embodiment, the sample is blood sample. The term “blood sample” means any blood sample derived from the subject. Peripheral blood is preferred, and mononuclear cells (PBMCs) are the preferred cells. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art.

As used herein, the term “extracting the miRNAs” from a biological sample refers to the purification of RNA from biological samples.

In a particular embodiment, the total RNA is prepared from blood, hippocampus, sperm according to published procedures⁴². In a particular embodiment, the total RNA is obtained from sperm. Typically, Sperm cells were isolated from ejaculation (man) and the epididymis (mice), and floating sperm were recovered by successive washing (PBS) and centrifugation at 3000 rpm. Briefly, RNAs were extracted from mice all tissues (hippocampus, blood and sperm cells) via a standard protocol. To remove the remaining DNA, the PCR samples were treated with DNase according to the manufacturer's instructions. Finally, the quantity (absorbance at 260 nm) and quality (ratio of absorbance at 260 nm and 280 nm) of the RNA were evaluated with a BioSpec-Nano spectrophotometer.

The clear supernatant is collected in 200 μl aliquots in new RNase/DNase-free microfuge tubes. RNA was isolated using a High Pure miRNA Isolation Kit (Cat. No: 5080576001; Roche, Mannheim, Germany) according to the manufacturer's instructions and stored at −80° C. until use. Isolated RNA samples were reverse transcribed into cDNA in 5 μl final reaction volumes using a TaqMan microRNA reverse transcription kit.

In a further embodiment, the quantity (absorbance at 260 nm) and quality (ratio of absorbance at 260 nm and 280 nm) of the RNA were evaluated with a BioSpec-Nano spectrophotometer.

As used herein the term “nucleic acid” has its general meaning in the art and refers to a coding or non-coding nucleic sequence. Nucleic acids include DNA (deoxyribonucleic acid) and RNA (ribonucleic acid). Example of nucleic acid thus include but are not limited to DNA, mRNA, tRNA, rRNA, tmRNA, miRNA, piRNA, snoRNA, and snRNA. The term “nucleic acid” also relates to circulating miRNA confined within exosomes). According to the invention, the term “nucleic acid” refers to nucleic acids present in the blood, serum, plasma or follicular fluid sample. The term “nucleic acid” also relates to nucleic acids originate from the brain that might go into the blood circulation. Any methods well known in the art may be used by the skilled artisan in the art for extracting the miRNA from the prepared sample (e.g. spectrometric methods, guanidinium thiocyanate-phenol-chloroform extraction, agarose gel electrophoresis, etc). For example, the method described in the example may be used.

As used herein, the term “miR” also known as micro RNA refers to single-stranded RNA approximately 20 to 25 nucleotides in length, more generally 21 to 24 nucleotides. miRNAs are repressors which act after transcription of a gene into mRNA: in fact, on pairing with messenger RNA, they guide their degradation or repress their translation into protein. The miRNA sequence publicly are available from the data base http://microrna.sanger.ac.uk/sequences/.

The production of miRNAs is under the tight control of a transcriptional and post-transcriptional regulation. The genes of miRNA are transcribed by RNA polymerase II into the form of long primary transcripts or precursors known as “pri-miRNA”. The precursor miRNAs are cleaved enzymatically in the nucleus of the cell by a class 2 RNAase III (Drosha) to form “pre-miRNA”s. “Pre-miRNA” is a RNA with a length of approximately 70 nucleotides, folded into an imperfect stem-loop by base pairing between the first half and the second half of its sequence. The pre-miRNAs are then exported to the cytoplasm where they bind with another nuclease (Dicer) and the RISC complex (RNA-induced silencing complex) which contains the proteins TRBP (transactivation-responsive RNA binding protein) and Ago2 (Argonaute 2). Upon binding, the protein Ago2 cleaves the 3′ ends of the miRNA precursor, thereby generating the mature miRNA duplex. Only the specific strand of the target mRNA of the miRNA is retained (thermodynamic reaction) in the complex; the other strand is removed and degraded.

As used herein, the term “hsa-miRxx” is the denomination for miRNAs identified in human (hsa meaning Homo sapiens). The sequences for all of the known miRNAs are recorded in bases such as miRBase or microRNAdb, where they are identified by their unique accession number (xx). In the list below, the number hsa-miRxx is used to refer to the mature miRNA sequence.

As used herein, the term “miR-19a-3p” also known as hsa-miR-19a-3p has the following sequence: UGUGCAAAUCUAUGCAAAACUGA (SEQ ID NO: 1 and Accession number: MIMAT0000073).

As used herein, the term “miR-361-5p” also known as hsa-miR-361-5p has the following sequence: UUAUCAGAAUCUCCAGGGGUAC (SEQ ID NO: 2 and Accession number: MIMAT0000703).

As used herein, the term “miR-3613-3p” also known as hsa-miR-3613-3p has the following sequence: ACAAAAAAAAAAGCCCAACCCUUC (SEQ ID NO: 3 and Accession number: MIMAT0017991).

As used herein, the term “miR-150-5p” also known as hsa-miR-150-5p has the following sequence: UCUCCCAACCCUUGUACCAGUG (SEQ ID NO: 4 and Accession number: MIMAT0000451).

As used herein, the term “miR-126-3p” also known as has-miR-126-3p has the following sequence: UCGUACCGUGAGUAAUAAUGCG (SEQ ID NO: 5 and Accession number: MIMAT0000445).

As used herein, the term “miR-499a-5p” also known as has-miR-499a-5p has the following sequence: UUAAGACUUGCAGUGAUGUUU (SEQ ID NO: 6 and Accession number: MIMAT0002870).

In a particular embodiment the method of the invention comprises the steps consisting of i) determining the level of at least one miR-19a-3p, miR-126-3p, miR-499a-5p, miR-361-5p, miR-3613-3p and/or miR-150-5p in the nucleic acid extraction, ii) comparing the level determined at step i) with a reference value (i.e. controls, especially healthy genitors and siblings), and iii) concluding that the subject is at risk to suffer from ASD when the level determined at step i) is lower than the reference value, or concluding that the subject is not at risk to suffer from ASD when the level determined at step i) is the same value as its predetermined reference value (controls and especially healthy genitors and siblings).

In a particular embodiment the method of the invention comprises the steps consisting of i) determining the level of at least one miR-19a-3p, miR-361-5p, miR-3613-3p, miR-150-5p, miR-126-3p, and/or miR-499a-5p in the nucleic acid extraction, ii) comparing the level determined at step i) with a reference value (i.e. controls, especially healthy genitors and siblings), and iii) concluding that the subject is at risk to suffer from ASD when the level determined at step i) is lower than the reference value, or concluding that the subject is not at risk to suffer from ASD when the level determined at step i) is the same value as its predetermined reference value (i.e. controls, especially healthy genitors and siblings).

In some embodiment, the reference value refers to the control, especially the healthy genitors and siblings controls.

In a particular embodiment, the reference value is a threshold value or a cut-off value that can be determined experimentally, empirically, or theoretically. A threshold value can also be arbitrarily selected based upon the existing experimental and/or clinical conditions, as would be recognized by a person of ordinary skilled in the art. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data.

Preferably, the person skilled in the art may compare the nucleic acid levels (obtained according to the method of the invention) with a defined threshold value. In one embodiment of the present invention, the threshold value is derived from the nucleic acid levels (or ratio, or score) determined in a blood, serum or plasma sample derived from one or more patients undergoing IVF or ISCI. Furthermore, retrospective measurement of the nucleic acid levels (or ratio, or scores) in properly banked historical blood, serum or plasma samples of patients undergoing IVF or ISCI may be used in establishing these threshold values.

As used herein, the term “same value” refers to maintain of level of miRNAs of the invention. Typically, the level of miRNAs of the invention is not significantly increased. Accordingly, when the level of miRNAs of the invention remains stable or increases not significantly, it means that the subject is not suffering from ASD. In the contrary, when the level of miRNAs of the invention is decreased, it means that the subject is suffering from ASD.

In a particular embodiment, the reference value refers to a control value. More particularly, the reference value is 1 for the six miRNA in human healthy sperm or blood samples.

In another embodiment, the reference value is 0.373±0.37; 0.344±0.34 or 0.380±0.38 for miR-3613-3p (child, father and mother controls respectively).

In another embodiment, the reference value is 10.570±7.06; 8.196±4.06 or 9.062±3.6 for miR-150-5p (child, father and mother controls respectively).

In another embodiment, the reference value is 0.066±0.03; 0.058±0.03 or 0.055±1.56 for miR-126-3p (child, father and mother controls respectively).

In another embodiment, the reference value is 0.103±0.08; 0.140±0.05 or 0.139±0.05 for miR-361-5p (child, father and mother controls respectively).

In another embodiment, the reference value is 0.086±0.05; 0.114±0.04 or 0.107±0.04 for miR-499a-5p (child, father and mother controls respectively).

In another embodiment, the reference value is 0.817±0.51; 1.069±0.47 or 1.009±0.45 for miR-19a-3p (child, father and mother controls respectively).

TABLE A
values of miRNA in patients and controls

| | miR- | miR- | miR- | miR- | miR- | miR- |
|---|---|---|---|---|---|---|
| | 3613-3p | 150-5p | 126-3p | 361-5p | 499a-5p | 19a3p |
| Patients | 0.069 ± | 1.900 ± | 0.001 ± | 0.001 ± | 0.001 ± | 0.182 ± |
| | 0.05 | 1.22 | 0.25 | 0.05 | 0.002 | 0.09 |
| Sibling of | 0.130 ± | 1.458 ± | 0.002 ± | 0.002 ± | 0.001 ± | 0.383 ± |
| Patients | 0.22 | 1.49 | 0.30 | 0.002 | 0.002 | 0.24 |
| Father of | 0.150 ± | 3.864 ± | 0.044 ± | 0.056 ± | 0.053 ± | 0.428 ± |
| Patients | 0.15 | 3.84 | 2.99 | 0.11 | 0.09 | 0.48 |
| Mother of | 0.190 ± | 4.844 ± | 0.046 ± | 0.080 ± | 0.065 ± | 0.574 ± |
| Patients | 0.19 | 3.74 | 0.12 | 0.06 | 0.12 | 0.43 |
| Child | 0.373 ± | 10.570 ± | 0.066 ± | 0.103 ± | 0.086 ± | 0.817 ± |
| Controls | 0.37 | 7.06 | 0.03 | 0.08 | 0.05 | 0.51 |
| Father | 0.344 ± | 8.196 ± | 0.058 ± | 0.140 ± | 0.114 ± | 1.069 ± |
| Controls | 0.34 | 4.06 | 0.03 | 0.05 | 0.04 | 0.47 |
| Mother | 0.380 ± | 9.062 ± | 0.055 ± | 0.139 ± | 0.107 ± | 1.009 ± |
| Controls | 0.38 | 3.6 | 1.56 | 0.05 | 0.04 | 0.45 |

Determination of the level of the nucleic acid can be performed by a variety of techniques well known in the art. In a particular embodiment, quantitative PCR may be performed for determining the level of DNA such as described in El Messaoudi et al., 2013; Mouliere et al., 2013; Thierry et al., 2013; Umetani et al., 2006 and WO2012/028746. In particular, the determination of the level of the nucleic acid may be performed by ALU-qPCR and techniques described in the examples.

In some embodiments, the present invention relates to an in vitro non invasive method for diagnosing ASD in a subject in need thereof comprising the steps consisting of i) providing a biological sample, ii) extracting the miRNAs from the biological sample and iii) determining the level of miR-19a-3p, miR-361-5p, miR-3613-3p, miR-150-5p, miR-126-3p, and/or miR-499a-5p in the nucleic acid extraction.

In a particular embodiment the method of the invention comprises the steps consisting of i) determining the level of miRNAs in the nucleic acid extraction, ii) comparing the level determined at step i) with a reference value, and iii) concluding that the subject has ASD when the level determined at step i) is lower than the reference value, or concluding that the subject has not ASD when the level determined at step i) is the same value as the reference value (i.e. controls, especially healthy genitors and siblings).

In a further aspect of the present invention, the levels of miRNAs are measured.

Accordingly, the method according to the present invention also comprises the step of determining the level of at least one miRNA selected from the group consisting miR-19a-3p, miR-361-5p, miR-3613-3p, miR-150-5p, miR-126-3p, and miR-499a-5p in the nucleic acid extraction.

The method of the invention may further comprise a step consisting of comparing the expression level of at least one miRNA in the nucleic acid extraction with a reference value, wherein lower expression level of the miRNA in the nucleic acid extraction compared to the reference value is indicative of a subject who is suffering or is susceptible to suffer from ASD. In one embodiment, the same value as controls references of expression level of miR-19a-3p, miR-361-5p, miR-3613-3p, miR-150-5p, miR-126-3p, and/or miR-499a-5p is indicative of a subject who is not at risk of having ASD.

In one embodiment, lower expression level of miR-19a-3p, miR-361-5p, miR-3613-3p, miR-150-5p, miR-126-3p, and/or miR-499a-5p is indicative of a subject who is at risk of having ASD.

In another embodiment, the invention is suitable to determine whether a subject is at risk to give birth to ASD children.

Accordingly, the invention relates to a method for determining whether a subject is at risk to give birth to ASD children comprising the steps consisting of i) determining the level of at least one miR-19a-3p, miR-126-3p, miR-499a-5p, miR-361-5p, miR-3613-3p and/or miR-150-5p in the nucleic acid extraction, ii) comparing the level determined at step i) with a reference value, and iii) concluding that the subject is at risk to give birth to ASD children when the level determined at step i) is lower than the reference value, or concluding that the subject is not at risk to give birth to ASD children when the level determined at step i) is the same value as the reference value.

In the context of the invention, the subject refers to a couple (a man and a woman) who wish to have a children.

### Method for Treating Autism Spectrum Disorder (ASD)

In a second aspect, the invention relates to a method of treating ASD in a subject in need thereof comprising a step of administering to the subject a therapeutically effective amount of a compound and/or conditions which increase the expression level of at least one miR-19a-3p, miR-126-3p, miR-499a-5p, miR-361-5p, miR-3613-3p and/or miR-150-5p.

The method according to the invention comprises i) a first step consisting in determining whether the subject suffers from ASD according to the method as described above, and ii) administering to the subject a therapeutically effective amount of a compound and/or conditions which increase the expression level of miR-19a-3p, miR-361-5p, miR-3613-3p, miR-150-5p, miR-126-3p, and/or miR-499a-5p when the level of miR-19a-3p, miR-361-5p, miR-3613-3p, miR-150-5p, miR-126-3p, and/or miR-499a-5p is lower than its predetermined reference value (i.e. controls, especially healthy genitors and siblings).

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder or to parents (in case they wish to have child) in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” refers to any mammals, such as a rodent, a feline, a canine, and a primate. Particularly, in the present invention, the subject is a human. In a particular embodiment, the subject is a human who is susceptible to have ASD. Typically, said subject has or is susceptible to have stereotypic behaviours and deficits in communication and social interaction. In the context of the invention, the subject is identified as having a lower level of miR-19a-3p, miR-361-5p, miR-3613-3p, miR-150-5p, miR-126-3p, and/or miR-499a-5p than its predetermined reference value.

As used herein, the term “compound” refers to a natural or synthetic compound that has a biological effect to activate the expression of miR-19a-3p, miR-361-5p, miR-3613-3p, miR-150-5p, miR-126-3p, and/or miR-499a-5p.

In a particular embodiment, the compound which increases the expression level of miR-19a-3p, miR-361-5p, miR-3613-3p, miR-150-5p, miR-126-3p, and/or miR-499a-5p is an agomir.

As used herein, the term “agomir” also known as mimics refers to a class of chemically engineered oligonucleotides.

In another embodiment, the compound which increases the expression level of miR-19a-3p, miR-361-5p, miR-3613-3p, miR-150-5p, miR-126-3p, and/or miR-499a-5p is Clozapine. Clozapine has trade names Clozaril, Leponex, Versacloz, developed by Novartis and has the following CAS Number: 5786-21-0.

In another embodiment, the compound which increases the expression level of miR-19a-3p, miR-361-5p, miR-3613-3p, miR-150-5p, miR-126-3p, and/or miR-499a-5p is selected from the group consisting of but not limited to: antipsychotics (Risperdal, Abilify); antidepressants (Prozac, Luvox, Zoloft, Celexa); stimulants (Ritalin, Adderall); anticonvulsants (Depakote, Dilantin, Klonopin, Tegretol); Revia; Xanax; Effexor; or Anafranil.

In another embodiment, the compound which increases the expression level of miR-19a-3p, miR-361-5p, miR-3613-3p, miR-150-5p, miR-126-3p, and/or miR-499a-5p is sugar.

In another embodiment, the following conditions allow an increase of expression levels of miR-19a-3p, miR-361-5p, miR-3613-3p, miR-150-5p, miR-126-3p, and/or miR-499a-5p. Typically, such condition is selected from the group consisting of but not limited to: temperature, oxygen, healthy food, exercise, care, altruism and kindness.

As used herein the terms “administering” or “administration” refer to the act of injecting or otherwise physically delivering a substance as it exists outside the body (e.g., agomirs of miR-19a-3p, miR-361-5p, miR-3613-3p, miR-150-5p, miR-126-3p, and/or miR-499a-5p or a compound which increases the expression level said miR) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of agomirs of miR-19a-3p, miR-361-5p, miR-3613-3p, miR-150-5p, miR-126-3p, and/or miR-499a-5p or a compound which increases the expression level said miR for use in a method for the treatment of ASD at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Composition for Treating Autism Spectrum Disorder (ASD)

In a third aspect, the invention relates to a pharmaceutical composition comprising a compound which increases the expression level of miR-19a-3p, miR-361-5p, miR-3613-3p, miR-150-5p, miR-126-3p, and/or miR-499a-5p.

In a particular embodiment, the pharmaceutical composition according to the invention for use in the treatment.

The pharmaceutical composition according to the invention for use in the treatment of ASD. The compound which increases the expression level of miR-19a-3p, miR-361-5p, miR-3613-3p, miR-150-5p, miR-126-3p, and/or miR-499a-5p as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention could be preparation of exosomes vesicles. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringe ability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxy propyl cellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatine. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Kit for Diagnosing Autism Spectrum Disorder (ASD)

In a fourth aspect, the present invention relates to a kit for performing the method according to the invention, wherein said kit comprises (i) means for determining the level of the miR-19a-3p, miR-361-5p, miR-3613-3p, miR-150-5p, miR-126-3p, and/or miR-499a-5p in a biological sample obtained from a subject who is suffering or is susceptible to suffer from ASD and/or at risk to have an ASD children and (ii) instructions for comparing with a reference value (i.e. controls, especially healthy genitors and siblings).

Typically, the kit includes primers, probes, macroarrays or microarrays. The kit may further comprise hybridization reagents or other suitably packaged reagents and materials needed for the particular hybridization protocol, including solid-phase matrices, if applicable, and standards. Alternatively the kit of the invention may comprise amplification primers that may be pre-labelled or may contain an affinity purification or attachment moiety. The kit may further comprise amplification reagents and also other suitably packaged reagents and materials needed for the particular amplification protocol.

The invention will be further illustrated by the following figures and examples. However, these examples and figures should not be interpreted in any way as limiting the scope of the present invention.

### FIGURES

FIG. 1. Decreased expression of the six-miRNAs miR-3613-3p, miR-150-5p, miR-126-3p, miR-361-5p, miR-19a-3p, and miR-499a-5p in children with autism and their fathers and mothers compared to age- and sex-matched healthy controls. In the cohort of patients with autism assembled in Kayseri, Turkey, we performed qRT-PCR as described in the Methods to analyze the microRNA profiles of serum samples from 45 autistic children (2-13 years old, 31 boys and 14 girls), their 33 siblings (1-20 years old, 17 boys and 16 girls), their 74 parents (mothers 23-45 years old, fathers 24-51 years old) and 21 control age-matched healthy children (3-16 years old, 10 boys and 11 girls) and their 16 parents, for a total of 189 participants. FIG. 1a The expression levels of six-miRNAs miR-3613-3p (A), miR-150-5p (B), miR-126-3p (C), miR-361-5p (D), miR-19a-3p (E), and miR-499a-5p (F) showed statistically significant differences (p<0.05). FIG. 1b the six-miRNA-transcript profiles graphed for a multiplex family (with more than one child with autism), and the tables below show the log fold-change rates for children with autism to control children (G, H) and their mothers (I, J) and fathers to father control (K, L) compared to age- and sex-matched healthy controls (p<0.0001). M. Heatmap shows the fold-change variation according to color, and in the figure, the columns represent the groups, and the rows represent the miRNAs (red, black and green correspond to upregulated, unchanged and downregulated, respectively).

FIG. 2. Family profiles for six-miRNAs miR-3613-3p, miR-150-5p, miR-126-3p, miR-361-5p, miR-19a-3p, and miR-499a-5p with a father of three behaviorally affected children. A-B. Family profiles, who has more than one affected child (girl is ASD, one of boys is schizophrenia), shows six-miRNAs compared to their healthy controls. C-E. Family profiles, who has an affected child, shows six-miRNAs compared to their healthy controls.

FIG. 3. Summary of animals, experiments and timeline. Whole groups (n=10) adult mice (2 months old, Balb c line) weighting 25-27 g on the test day was conducted behavioral test novel object, social behavioral test, Marble burying test and tail suspension test respectively. *p<0.05, **p<0.001, ****p<0.0001. After behavioral test, whole groups of mice (n=5) was sacrificed and blood, hippocampus and sperm samples were harvested.

VPA (Valproic Acid)

Cc2d1a Group

G1=Cc2d1a^{+/-}×Cc2d1a^{+/+}, G2=Cc2d1a^{+/-}×Cc2d1a^{+/-}, control=Cc2d1a^{+/+}×Cc2d1a^{+/+} crosses are presented.

A—Novel object recognition (NOR) test. Time spent with familiar versus novel objects (see Materials and Methods). Object recognition is measured as the difference in the time spent with the familiar versus the new objects. A-Valproic acid-treated males (500 mg/kg dose).

B—Cc2d1a group, offspring from G1=Cc2d1a^{+/-}×Cc2d1a^{+/+}, G2=Cc2d1a^{+/-}×Cc2d1a^{+/-}, control Cc2d1a^{+/+}×Cc2d1a^{+/+} crosses are presented. Controls for all groups are normal wild type Cc2d1a^{+/+}Balb c, that never been crossed with the mutants mice.

Social Interaction Test

To determine whether the interactions varied between groups of mice, the mice were placed in the center of a cage divided into three compartments, where chamber A contained another mouse, and chamber B was empty (see Materials and Methods). They were filmed for 5 minutes to calculate the time that they spend close to the empty compartment or to the compartment occupied by another mouse. C-Valproic acid-treated males (mg/kg dose). D-Cc2d1a group males, offspring from G1=Cc2d1a^{+/-}×Cc2d1a^{+/+}, G2=Cc2d1a^{+/-}×Cc2d1^{+/-}, and control=Cc2d1a^{+/+}×Cc2d1a^{+/+} crosses are presented. Controls for all groups are normal wild type Balb c, that never been crossed with the mutants mice.

Tail Suspension Test

The test consists of short-term suspension of mice by their tail (six minutes here). They were filmed and analyzed for immobility posture (see Materials and Methods). E-Valproic acid-treated males. F—CC2d1a group males, offspring from G1=Cc2d1a^{+/-}×Cc2d1a^{+/+}, G2=Cc2d1a^{+/-}×Cc2d1a^{+/-}, and control=Cc2d1a^{+/+}×Cc2d1a^{+/+} crosses are presented. Controls for all groups are normal wild type Cc2d1a^{+/+}, Balb c, that never been crossed with the mutants mice.

Glass Marble Burying Test

Repetitive action was evaluated via the marble burying test (see Materials and Methods). Twenty glass marbles were placed on the surface of clean bedding, and the number of marbles buried in 30 minutes was scored. G-Valproic acid-treated males. H—CC2d1a group males, offspring from G1=Cc2d1a^{+/-}×Cc2d1a^{+/+}, G2=Cc2d1a^{+/-}×Cc2d1a^{+/-}, control=Cc2d1a^{+/+}×Cc2d1a^{+/+} crosses are presented. Controls for all groups are normal wild type Cc2d1a^{+/+}, Balb c, that never been crossed with the mutants mice.

FIG. 4. miRNA expression profiles of mouse sperm. Total sperm RNAs from four-month-old Balb c males were tested by q-PCR for microRNAs. A in graph and B in fold change from VPA-treated males (500 mg/kg) are compared to those of controls. C in graph a for group and offspring genotypes produced by the following crosses: G1=Cc2d1a^{+/-}×Cc2d1a^{+/+}, G2=Cc2d1a^{+/-}×Cc2d1a^{+/-}, and control=Cc2d1a^{+/+}×Cc2d1a^{+/+}. Fold-change values for six differentially expressed miRNAs are illustrated by the variation in color for the 5p strand (6).

FIG. 5. Sperm profiles of the six-miRNAs in the father of three affected children. A-D. Sperm profiles of P13F (raw data) is presented for six-miRNAs in a father patient and the three controls. Total sperm RNA was tested by qRT-PCR to determine the levels of the Six-microRNAs. The miRNA transcript profile of human sperm from P13F (the father of three children with distinct behavioral alterations born to different mothers) compared to three sperm sample controls is shown.

### MATERIAL & METHODS

Patient Selection Criteria

This study was approved by the Ethics Committee of the Erciyes University School of Medicine. A detailed description of the study was provided to all participants and their parents before their enrollment. All parents gave written informed consent before participation (09-20-2011 committee number: 2011/10). The diagnosis was made by a multidisciplinary team (composed of an experienced child psychiatrist, a pediatric neurologist, and a genetic specialist) according to the criteria of the Diagnostic and Statistical Manual, Fourth and Fifth Edition, Text Revision (DSM-IV-TR; American Psychiatric Association, 2000 and DSM-V; American Psychiatric Association, 2013) using the Childhood Autism Rating Scale (CARS) (Schopler, Reichler, DeVellis, & Daly, 1980). All subjects were carefully screened for signs of infection, and subjects with acute illness were excluded. Nine multiplex families (including more than 1 child with an autism diagnosis) and 28 simplex families (including only 1 child with autism) were enrolled in this study. A total of 189 participants were enrolled in the study. We included 45 subjects with ASD (range=2 to 13 years old; 31 males and 14 females) and 21 age- and sex-matched typical control subjects (range=3 to 16 years old; 10 males and 11 females). Thirty-three healthy siblings (range=1 to 20 years old; 17 males and 16 females) were included as well as their parents. All of the participants were of Turkish origin. The ASD group included 27 children with autism spectrum disorder and 18 children with pervasive developmental disorder-not otherwise, specified (PDD-NOS). Twenty-two of the children were diagnosed with intellectual disability, 4 of whom were diagnosed with epilepsy with EEG abnormalities, and 2 of them also exhibited attention deficit hyperactivity disorders (ADHDs) among the autistic cases. Eleven children were diagnosed with mental retardation, 5 of whom were diagnosed with ADHD among the PDD-NOS cases. Patients were excluded if they exhibited genetic disorders, including chromosomal abnormalities, Fragile X Syndrome, tuberous sclerosis, or neurofibromatosis type 1. No clinical or laboratory findings suggesting autism or other diseases were detected in the control group.

Blood Collection, Serum Separation and RNA Isolation

Blood samples were collected from the donors and healthy controls after obtaining written informed consent from all of the parents. In total, 189 family members (45 autistic patients, 33 unrelated healthy siblings, and 74 parents) and 37 sex- and age-matched healthy control samples were included in the dynamic array to investigate 384 miRNAs. Two-milliliter blood samples were collected from all of the family members and matched controls. Blood samples were collected between 11.00 and 13.00 to eliminate unwanted variation in the examined parameters. All protocols for serum separation were completed within 1 hour of drawing blood. Then, the samples were held for 30 minutes at room temperature. Serum was separated by centrifugation at 3500 rpm for 10 minutes at room temperature. Hemolyzed samples were excluded from the study. The clear supernatant was collected in 200 μl aliquots in new RNase/DNase-free microfuge tubes. RNA was isolated using a High Pure miRNA Isolation Kit (Cat. No: 5080576001; Roche, Mannheim, Germany) according to the manufacturer's instructions and stored at −80° C. until use.

cDNA Preparation and Pre-Amplification

Isolated RNA samples were reverse transcribed into cDNA in 5 μl final reaction volumes using a TaqMan microRNA reverse transcription kit (Applied Biosystems, Foster City, CA, USA) as specified in the manufacturer's protocol. Reverse transcription was performed using a LightCycler 480 Real-Time PCR System (Roche, Mannheim, Germany). cDNA samples were kept at −80° C. until PCR analysis. We performed preamplification after reverse transcription using a TaqMan PreAmp Master Mix 29 system (Applied Biosystems, Foster City, CA, USA) as well as the Megaplex Human Primer Pools Set v3.0 (Applied Biosystems, Foster City, CA, USA). For preamplification, 2 μl of a 1/5-diluted RT product was added to 3 μl of the PreAmp mix. The miRNA TaqMan PreAmp thermal protocol was as follows: 95° C. for 600 sec, 55° C. for 120 sec and 72° C. for 120 sec, followed by 18 cycles of 95° C. for 15 sec and 60° C. for 240 sec, and finally, 99.9° C. for 600 sec. The preamplified cDNA samples were stored at −20° C. for further analysis.

Quantitative Real-Time Polymerase Chain Reaction (qRT-PCR)

qRT-PCR was performed by using a high-throughput BioMark Real-Time PCR system (Fluidigm, San Francisco, CA, USA). Preamplified cDNA samples were diluted with low-EDTA (0.1 mM) TE buffer (1:5). Approximately 490 μl of TaqMan Universal PCR Master Mix, No AmpErase UNG (Applied Biosystems, Foster City, CA, USA) and 49 μl of 20×GE Sample Loading Reagent (Fluidigm, San Francisco, CA, USA) were mixed and pipetted into a 96-well plate, and 3.85 and 3.15 μl of 1:10-diluted preamplified cDNA was pipetted into each well and mixed. Then, 5 μl of this mixture was pipetted into the sample inlets of a 96.96 Dynamic Array (Fluidigm, San Francisco, USA), and 4 μl aliquots of 1:1-diluted 209 Assays were pipetted into the assay inlets of a 96.96 Dynamic array (Fluidigm, San Francisco, USA). A BioMark IFC controller HX (Fluidigm, San Francisco, CA, USA) was used to distribute the assay mixture and the sample mixture from the loading inlets into the 96.96 Dynamic array reaction chambers for qRT-PCR by using Fluidigm's Integrated Fluidic Circuit Technology. The real-time PCR step was performed by using a BioMark System with the following protocol: the thermal mixing protocol was followed by heating at 50° C. for 120 sec, 70° C. for 1,800 sec, and 25° C. for 600 sec. Then, the UNG and hot-start protocol were followed by heating at 50° C. for 120 sec and 95° C. for 600 sec. Finally, PCR was performed with 40 cycles at 95° C. for 15 sec and 60° C. for 60 sec. The BioMark system is quantifies low-abundance miRNAs and can detect a single copy at a Ct value of 26-27.

Routine qPCR for miR detection and validation⁴¹ was performed with the miScript PCR control set (catalog number 218380; Qiagen, Germany). The miScript™ miRNA PCR Array Human Serum & Plasma 384HC (Cat No: 331223) was used in this study. The Human Serum & Plasma 384HC miScript miRNA PCR Array profiles the expression of 372 miRNAs) that are detectable in serum and plasma using the miScript PCR system. SNORD61, SNORD68, SNORD72, SNORD95, SNORD96A, RNU6-2, miRTC, miRTC, and PPC were used as controls. The data were normalized using the 2^{-ΔΔct} method.

Data Normalization and Analysis

Data were collected using Fluidigm Real-Time PCR Analysis Software, the linear derivative baseline correction method, and the auto global Cq threshold method. System-provided Cq values of 999 and values larger than 26 were considered nonspecific and beyond the detection limits of the system and were therefore removed. Median LOD Cq values were calculated across all arrays to impute missing values. Data normalization was performed using the 2^{-ΔΔCT} method. To detect the differentially expressed miRNAs, linear models for the microarray and RNA-Seq data (limma) were applied. The Benjamini-Hochberg false discovery rate correction was used to adjust p values to account for multiple testing. DIANA-mirPath software (Vlachos, 2005) was used to identify signaling pathways based on the differentially expressed miRNAs. Highly accurate and experimentally verified TarBase targets were considered potential targets, and the agglomerative hierarchical clustering algorithm was applied to determine miRNA and pathway clusters based on their interaction levels. The analysis was conducted using the Rcmdr (Fox, J., 2005) and limma⁴¹ packages of R 3.1.2 software (URL http://www.R-project.org/.) and, p-values less than 5% were considered to indicate statistical significance.

Mice

The mice were maintained in a facility under controlled conditions (light from 06:00 to 18:00, 22° C. temperature, 55% humidity). All animal models are followed in the same animal house and time scale, well controlled conditions of food, water, temperature, light and care. Only two persons, always the same are allowed to enter to animal rooms. The animals were cared for and treated according to the Principles of Laboratory Animal Care (European rules). All experiments were approved by the Erciyes University Animal Ethics Committee (04-11-2012) (12/54). All tests were performed between 10:00 and 16:00 in isolated rooms. In all tests, the results for the Balb/c mouse line are presented.

Genotyping

Cc2d1a^{+/-} mice were purchased from the Jackson Laboratory. Heterozygotes were produced by outbreeding in the Balb c background for 10 generations before the initiation of the experimental procedures. Heterozygotes were selected by PCR genotyping according to the instructions for the corresponding Jackson Laboratory²⁹ oligonucleotides (CCD1A-M1: 5′-GTG CGA GGC CAG AGG CCA CTT CTG-3′ (SEQ ID NO: 7), CCD1A-M2: 5′-GAC CCT GAG AGA GCT CCT GAG AGC-3′ (SEQ ID NO: 8), CCD1A-M3: 5′-TT CCC ACC TCT TCT GGC CCA GAG G-3′ (SEQ ID NO: 9).

RNA Extraction

Total RNA was prepared from mouse tissues (blood, hippocampus, sperm) according to published procedures⁴². Sperm cells were isolated from the epididymis, and floating sperm were recovered by successive washing (PBS) and centrifugation at 3000 rpm. Briefly, RNAs were extracted from all tissues (hippocampus, blood and sperm cells) via a standard protocol. Qiazol Lysis Buffer (Cat. No: 79306; Qiagen, Texas, USA) was used in accordance with the manufacturer's instructions. The Qiazol-extracted aqueous phase was ethanol precipitated, followed by washing twice with 70% ethanol. To remove the remaining DNA, the PCR samples were treated with DNase according to the manufacturer's instructions.

Finally, the quantity (absorbance at 260 nm) and quality (ratio of absorbance at 260 nm and 280 nm) of the RNA were evaluated with a BioSpec-Nano spectrophotometer. RNA was stored at −80° C. until use.

VPA Postnatally Exposed Males

The sodium salt of VPA (Sigma, St. Louis, MO) was prepared in 0.9% saline at concentrations of 300, 400, 500, 600 and 700 mg/ml, pH 7.4 (data not shown). The control group received the same volume (0.1 ml) of 0.9% saline buffer. At 14 days post birth, all offspring (8-12 pups from each litter) were injected (intraperitoneally) with VPA at 300 to 700 mg/ml or with saline buffer as a control. Doses of 600 and 700 mg/kg resulted in dramatic lethality, and the animals were therefore not included in the test group. The F1 to F2 generations were obtained following treatment with the 300 to 500 mg/kg VPA doses. The F1 generation derived from VPA-injected F0 fathers is referred to as VPA-F1, while that from saline-injected fathers is identified as the control. Adult VPA-F1 males were bred with normal females.

The control groups were bred under the same housing conditions across all generations. Only male mice were utilized for all experiments.

Tests of Autism-Like Behaviors in Mouse Models (VPA-Treated Group and Cc2d1a Heterozygotes)

Novel Object Recognition Test

The time spent with familiar versus novel objects highlighted a common characteristic of the test group versus the controls. Specifically, it indicated differences in the interest in and recognition of objects. NOR (novel object recognition) is a well-established test in a variety of animal models with multiple protocols. In general, it involves two trials of cognition evaluation based on the spontaneous exploratory conduct of a mouse to measure recognition memory. In the first trial, we used (first-day acquisition) animals that were exposed to two similar objects (small orange boxes) in a chamber for 5 minutes. During the second trial (second-day retention) mice were again exposed to two dissimilar objects for 5 minutes, including one familiar object from the first trial and one new object (blue box). Object recognition was measured (data not shown), according to the difference in the time spent with the familiar object versus the new object.

Social Interaction Test

Mice grouped in cages exhibit additional behaviors that are referred to as social interactions. To determine whether these interactions varied between the groups of mice, they were filmed for 5 minutes. There are many different ways to follow social interaction. Here, we present the results in a simple way based on conditions in a cage separated into three sections. The mice being tested were placed in the center of the cage and were filmed to calculate the time that they spent close to the empty compartment or the compartment occupied by another mouse. The three chambers were separated by square doors in the two inner walls connecting to the center area. Briefly, the test mouse was introduced in the center to initiate habituation for 5 minutes while blocking access to the side compartments. The first test was conducted by placing an unfamiliar mouse inside an empty wire cage in one of the side chambers to measure the social interaction of the subject mouse without direct social contact. On the other side, there was an empty wire cage. Each test was performed for 10 minutes (data not shown), and the floor surfaces were wiped with 70% ethanol between trials. The accumulated time spent in each compartment and the sociability or social preference indices were measured to quantify the social behavior of the mice.

Tail Suspension Test

Adult mice (2 months old) (Balb c strain) weighing 25-27 g on the test day. The apparatus used for the tail test consisted of two filter covers each and enabled three mice to be tested simultaneously. Each mouse was suspended by the tail from a hook connected to the strain gauge, to which they were attached with adhesive tape with a length of 18 cm. The duration of each trial was 360 seconds. The tail suspension test was recorded using a SONY HDR-CX240E video recorder. After the recording, the period of the immobility of the mice (in which they remained inactive) was calculated manually. An immobility posture indicates the abandonment of struggling and, thus, depression. Periods of agitation and immobility are reported (data not shown).

Glass Marble Burying Test

According to the described protocols, newly filled bedding cages were prepared with 20 marbles spaced on the bedding surface. Mice were individually caged. After 30 minutes, each mouse was returned to its home cage, and the buried marbles were counted (data not shown). Marbles that were not buried were not included in the analysis.

F1 VPA Generation

The next generation of mice was obtained from crosses between VPA-treated males and normal partners. At two months, their behavior was tested and compared to that of the founder males. Variation was observed between the F1 mice and the father, but they were still different from the control group.

Statistical Analysis.

All data are presented as the mean and standard deviation (SD). For the statistical analysis of the behavioral test results, an unpaired t-test and one-way ANOVA were performed in the case of a significant comparison result (p<0.05), Tukey's test was used as a post hoc multiple comparison test). The p-value significance level was set at <0.05.

TABLE 1
Transcription values in different groups for “Six-miRNAs”. Transcription profiles of “Six-
miRNAs” (raw data after normalization) in children with autism and their siblings, mothers
and fathers compared to age- and sex-matched healthy controls (p < 0.0001) (Mann-Whitney tests)

| | | | | | | | | Log(fold |
|---|---|---|---|---|---|---|---|---|
| | | | | | | | Log(fold | change) |
| | | | | | | | change) | Mothers |
| | | | Log(fold | Log(fold | Log(fold | Log(fold | Patients | have more |
| | | Log(fold | change) | change) | change) | change) | have | than one |
| | Log(fold | change) | Mothers of | Fathers of | Female | Male | autistic | autistic |
| | change) | Patients | patients | patients | patients | patients | siblings | children |
| miRNAs | Control | (n = 45) | (n = 37) | (n = 37) | (n = 14) | (n = 31) | (n = 18) | (n = 9) |
| miR-3613- | 1 | −0.30436 | −0.03851 | −6.806063758 | −0.00677 | −0.0195 | −0.32272 | −0.10768 |
| 3p | | | | | | | | |
| miR-150- | 1 | −8.68965 | −1.89998 | −0.313461259 | 0.092496 | −0.47326 | −9.05134 | −3.48457 |
| 5p | | | | | | | | |
| miR-126- | 1 | −3.46494 | −0.02768 | −2.1599325 | 0.095035 | −0.00107 | −3.65435 | 3.989511 |
| 3p | | | | | | | | |
| miR499a- | 1 | −0.08505 | −0.03757 | −0.091225794 | 0.000171 | −0.00114 | −0.10867 | −0.06383 |
| 5p | | | | | | | | |
| miR-361- | 1 | −0.102 | −0.05434 | −0.066004089 | 0.000126 | −0.00084 | −0.08928 | −0.04199 |
| 5p | | | | | | | | |
| miR-19a- | 1 | −0.63541 | −036743 | −0.71661666 | −0.01343 | −0.042042 | −0.70797 | −0.50073 |
| 3p | | | | | | | | |

TABLE 2
Decreased expression level of the six-miRNAs. The
log fold change rates of six differentially expressed
miRNAs in the serum of the different groups.

| | miR- | miR- | miR- | miR- | miR- | miR- |
|---|---|---|---|---|---|---|
| | 3613-3p | 150-5p | 126-3p | 361-5p | 499a-5p | 19a3p |
| Patients | 0.069 ± | 1.900 ± | 0.001 ± | 0.001 ± | 0.001 ± | 0.182 ± |
| | 0.05 | 1.22 | 0.25 | 0.05 | 0.002 | 0.09 |
| Sibling of | 0.130 ± | 1.458 ± | 0.002 ± | 0.002 ± | 0.001 ± | 0.383 ± |
| Patients | 0.22 | 1.49 | 0.30 | 0.002 | 0.002 | 0.24 |
| Father of | 0.150 ± | 3.864 ± | 0.044 ± | 0.056 ± | 0.053 ± | 0.428 ± |
| Patients | 0.15 | 3.84 | 2.99 | 0.11 | 0.09 | 0.48 |
| Mother of | 0.190 ± | 4.844 ± | 0.046 ± | 0.080 ± | 0.065 ± | 0.574 ± |
| Patients | 0.19 | 3.74 | 0.12 | 0.06 | 0.12 | 0.43 |
| Child | 0.373 ± | 10.570 ± | 0.066 ± | 0.103 ± | 0.086 ± | 0.817 ± |
| Controls | 0.37 | 7.06 | 0.03 | 0.08 | 0.05 | 0.51 |
| Father | 0.344 ± | 8.196 ± | 0.058 ± | 0.140 ± | 0.114 ± | 1.069 ± |
| Controls | 0.34 | 4.06 | 0.03 | 0.05 | 0.04 | 0.47 |
| Mother | 0.380 ± | 9.062 ± | 0.055 ± | 0.139 ± | 0.107 ± | 1.009 ± |
| Controls | 0.38 | 3.6 | 1.56 | 0.05 | 0.04 | 0.45 |

Results

Altered Serum miRNA Profiles in a Cohort of 45 Autistic Patients.

A cohort of patients with autism, unaffected family members and healthy controls was assembled at the Erciyes University School of Medicine Hospital, Kayseri, Turkey (n=189). It comprised affected children (n=45, 2-13 years old 31 boys and 14 girls), their unaffected siblings (n=33, 1-20 years-old, 17 boys and 16 girls), their parents (n=74, mothers 23-45 years old, fathers 24-51 years old), control age-matched children (n=21, 3-16 years, 10 boys and 11 girls) and 16 control parents, among families 9 multiplex (more than one child with autism) and 28 simplex (one child with autism).

The serum miRNA expression profiles of all the family members (mothers, fathers, sisters, brothers and the autistic children) were compared to those of the age- and sex-matched healthy controls. MicroRNA analysis was carried out by qRT-PCR analysis of the serum samples of a total of 189 participants. The technique is described in the Methods section. Statistically significant results (p<0.05) were registered for 280 miRNAs in the children with autism and their families compared to the controls.

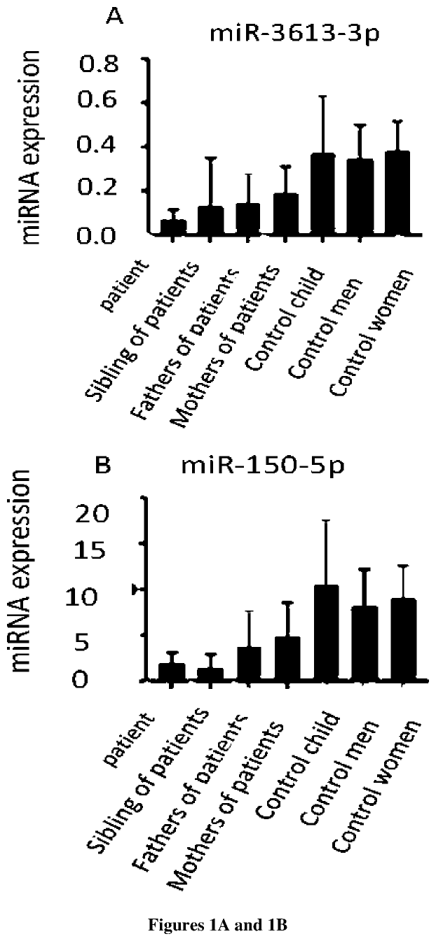

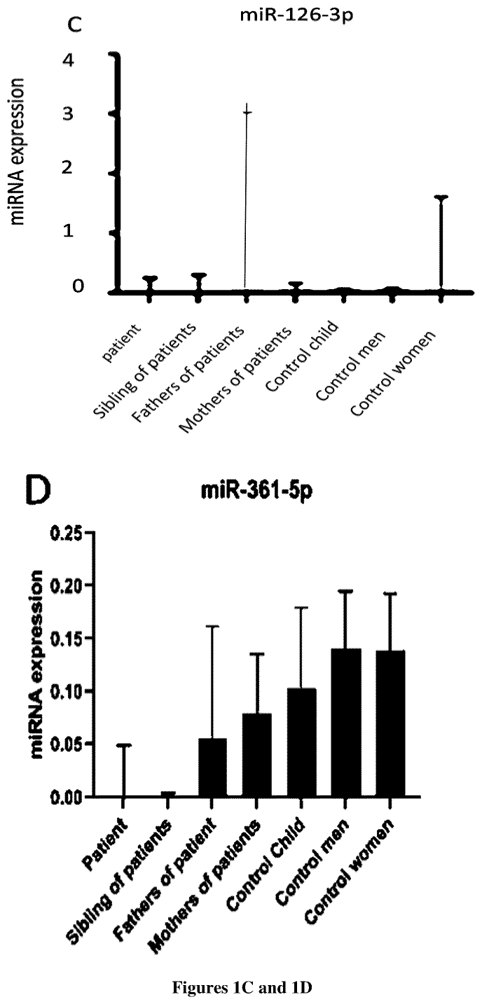

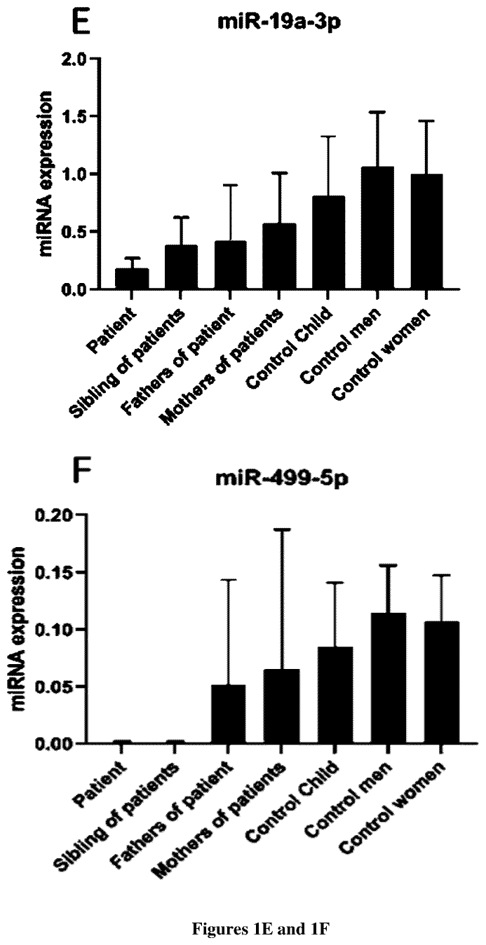

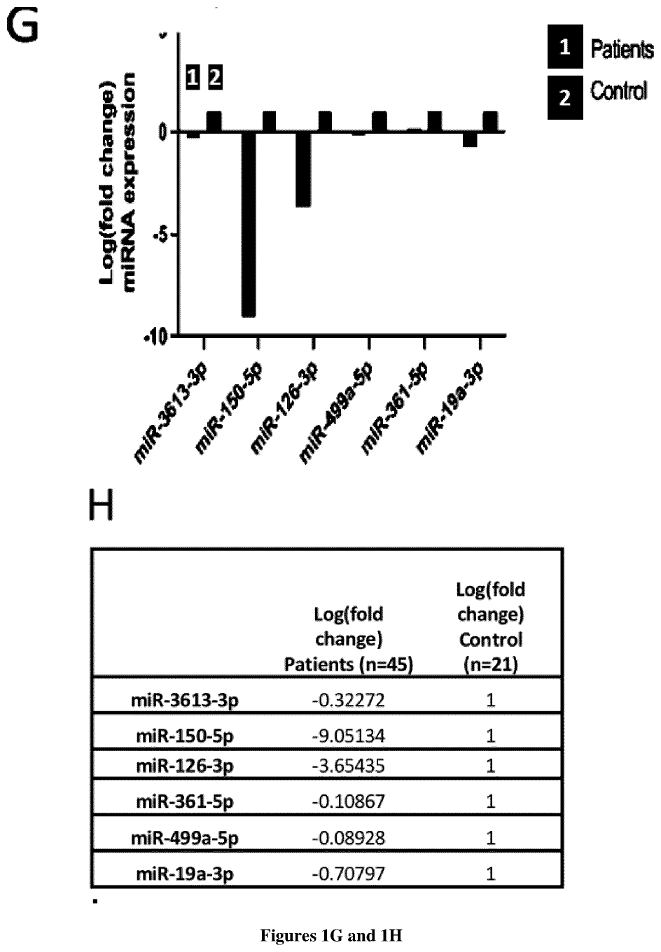

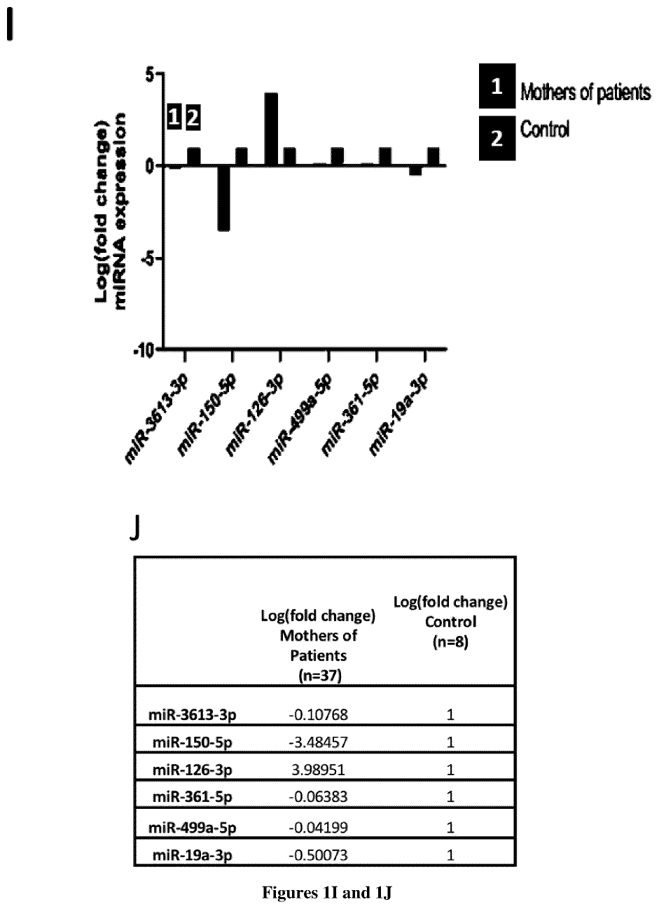

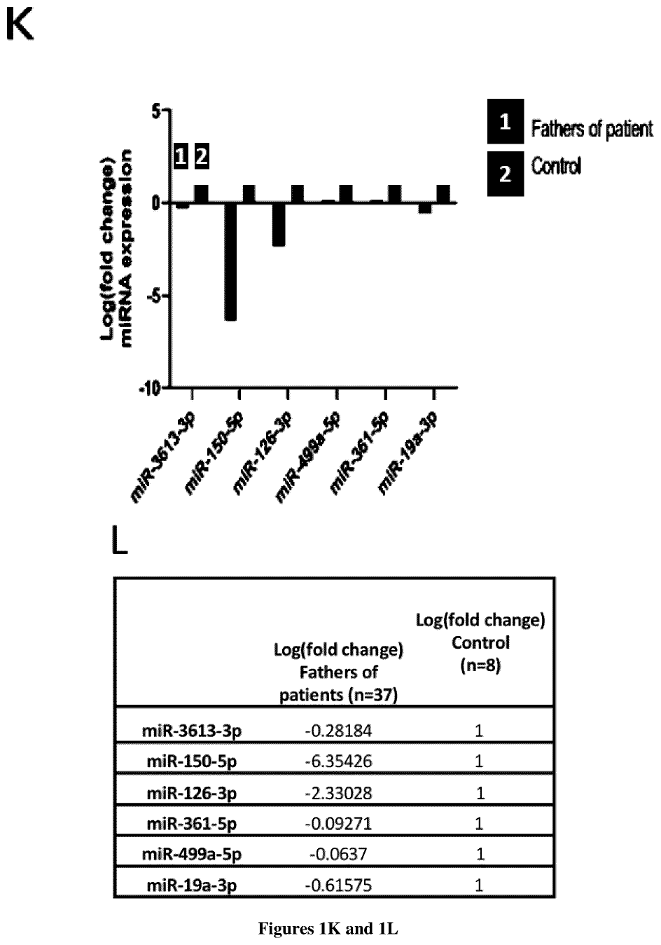

While most of the microRNAs tested (data not shown), exhibited normal (taken as 100 percent) to lower levels in the patients expression levels in the range of 1 to 10 percent of the healthy control level were noted for six of them in FIG. 1 and Table 1: miR-3613-3p (FIG. 1A), miR-150-5p (FIG. 1B), miR-126-3p (FIG. 1C), miR-361-5p (FIG. 1D), miR-19a-3p (FIG. 1E), miR-499a-5p (FIG. 1F) and in FIG. 1, Table 1 and. In FIG. 1 G to L are shown successively patients to control (FIG. 1G, H), mothers of patients to controls (FIG. 1 I, J) and fathers of patients to controls (FIG. 1K, L), patients and relatives with lower levels to controls groups. Table 2 shows fold change (numeric values) and inventors show the fold change values of the six-miRNAs differentially expressed in the serum of the different groups indicated by color (data not shown). FIG. 1M shows nine multiplex (more than one child with autism) to twenty-eight simplex (one child with autism) families. Non differences between two group (multiplex/simplex) in the levels of the six miRNAs were observed.

Intermediate Profiles of Apparently Healthy Relatives.

Intermediate levels were recorded for the same six-miRNAs in the serum of the healthy siblings, fathers and mothers of the affected children. Although the levels of these miRNAs were higher than in the affected children, they were significantly lower by 40 to 50 percent FIG. 1 and Table 1 (at least one of the parents) compared with those in genetically unrelated controls (FIG. 1 and data not shown). All comparisons are separately listed including those between (a) patient mothers and healthy mothers; (b) patient fathers and healthy fathers; (c) autism patients and healthy siblings; and (d) female autism patients and healthy female siblings (data not shown).

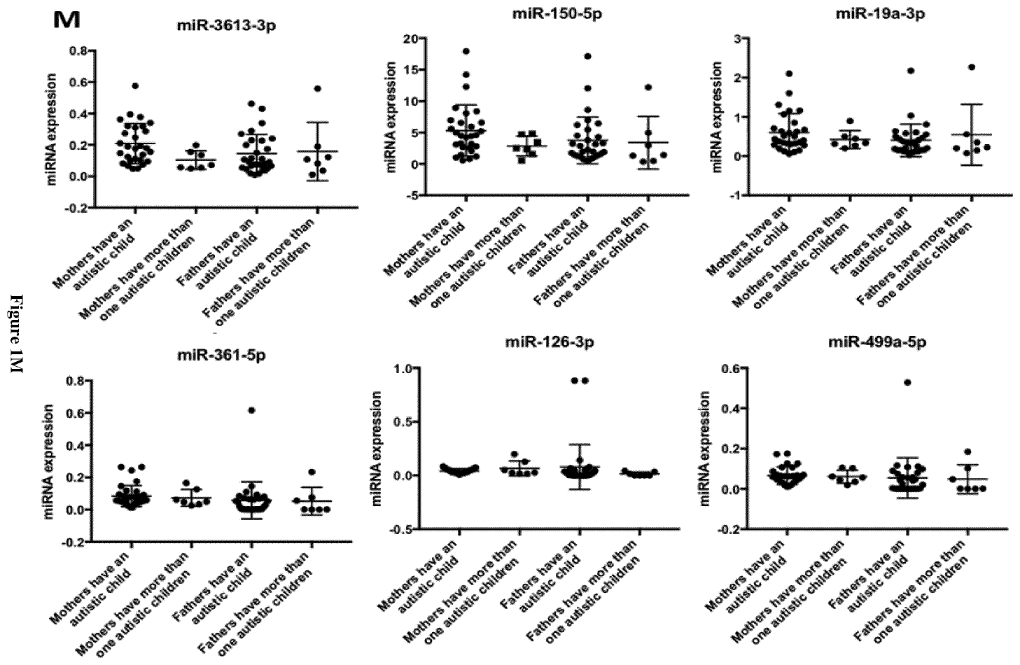

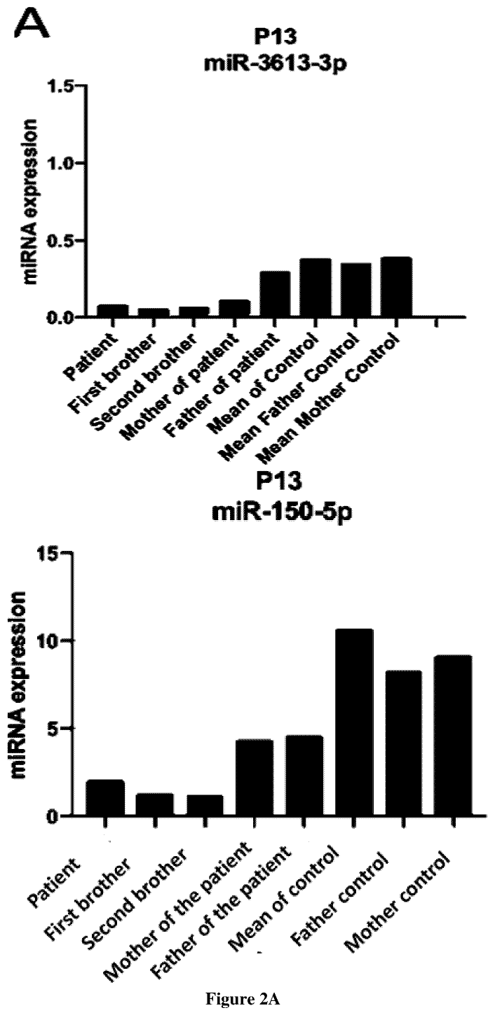

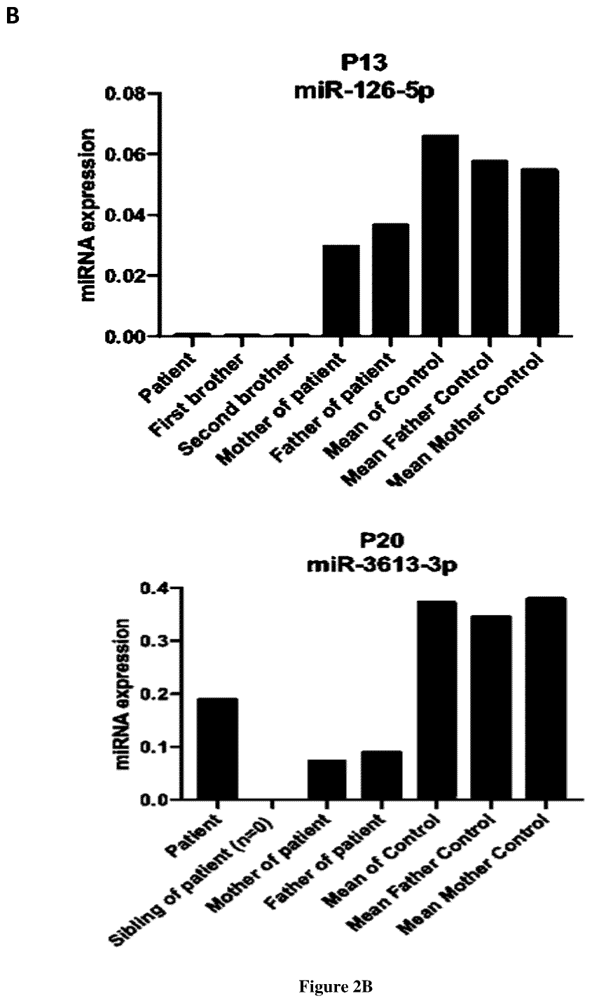

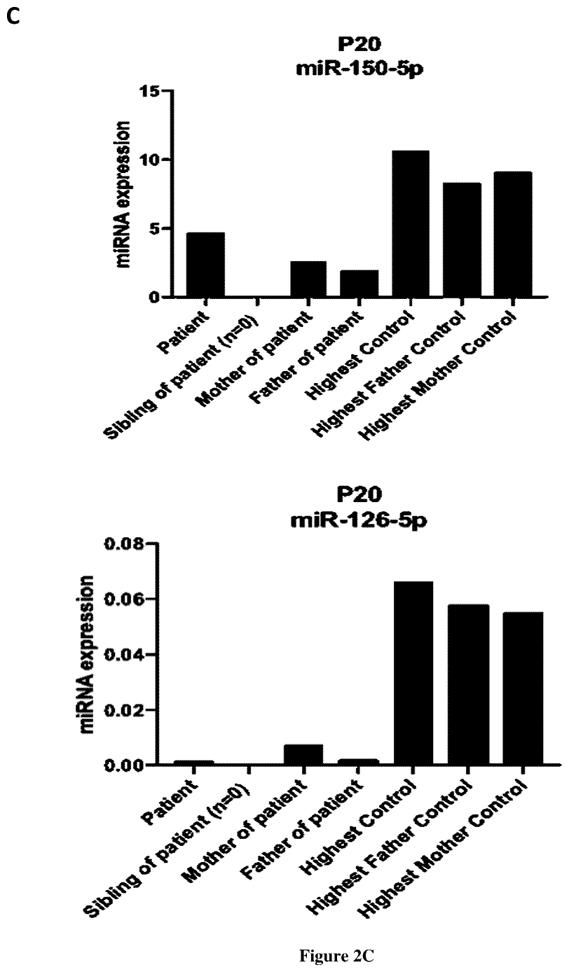

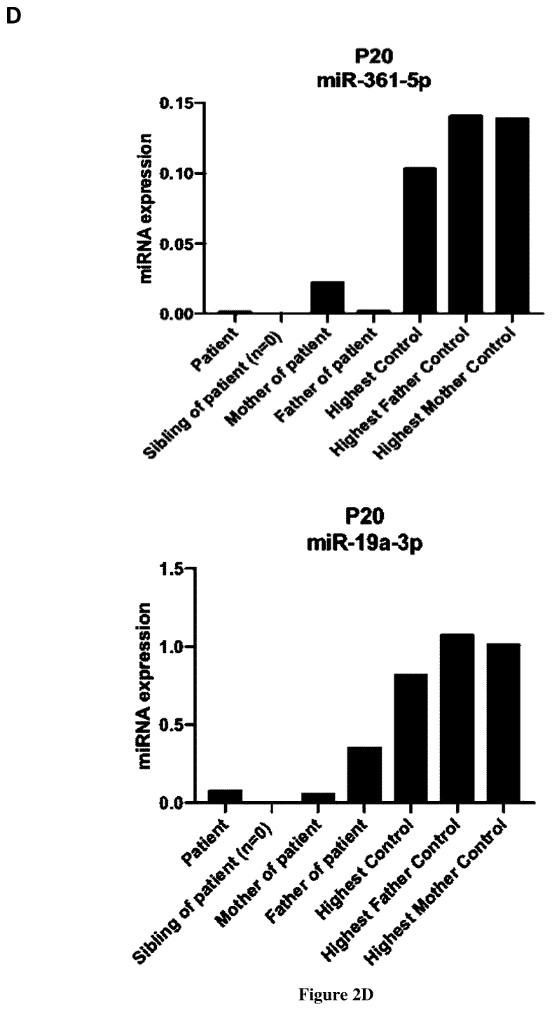

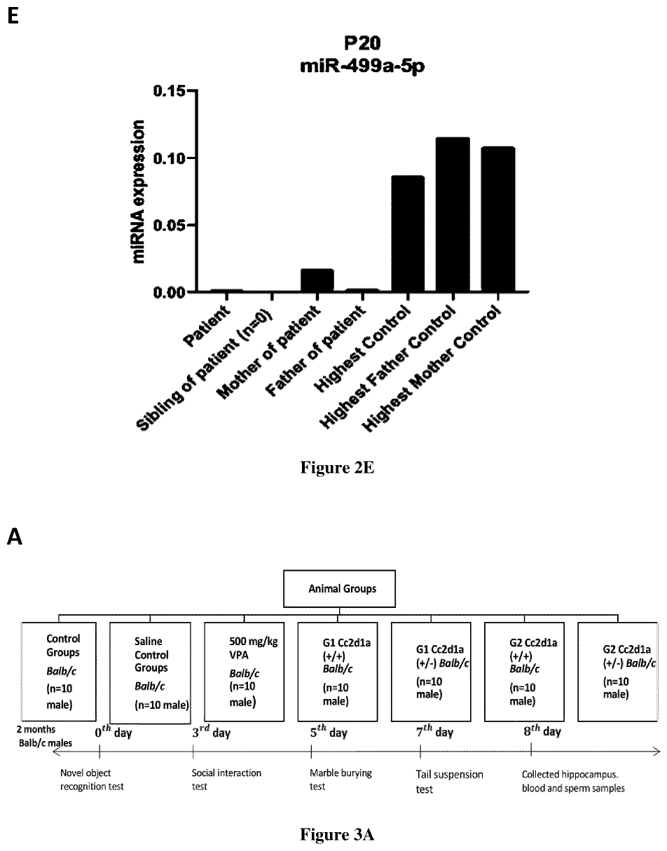

Individual Examples of Families with Either Three Affected Children (Multiplex) or One Affected Child (Simplex)—(FIG. 2A-2E).

Analyzing the distribution of the markers in large families with ASD and related diseases may be of interest for further studies. In FIG. 2 is shown one example in the present cohort, FIG. 2 A illustrates the case of a family with three affected children, including one girl autistic patient born to one mother and two boy patients with schizophrenia born to a different mother. All six-miRNAs down-regulated in patients presented low levels in the serum blood of daughter patient, her two half brothers to mother and father (FIG. 2 A). FIG. 2 B in comparison illustrates the case of a family with one affected child with the same results that presents lower level of six-miRNAs to parents and to controls.

Mouse Models of ASD (Autism Spectrum Disorders) Confirm the Reduced Levels of the Six microRNAs as Characteristic of the Disease.

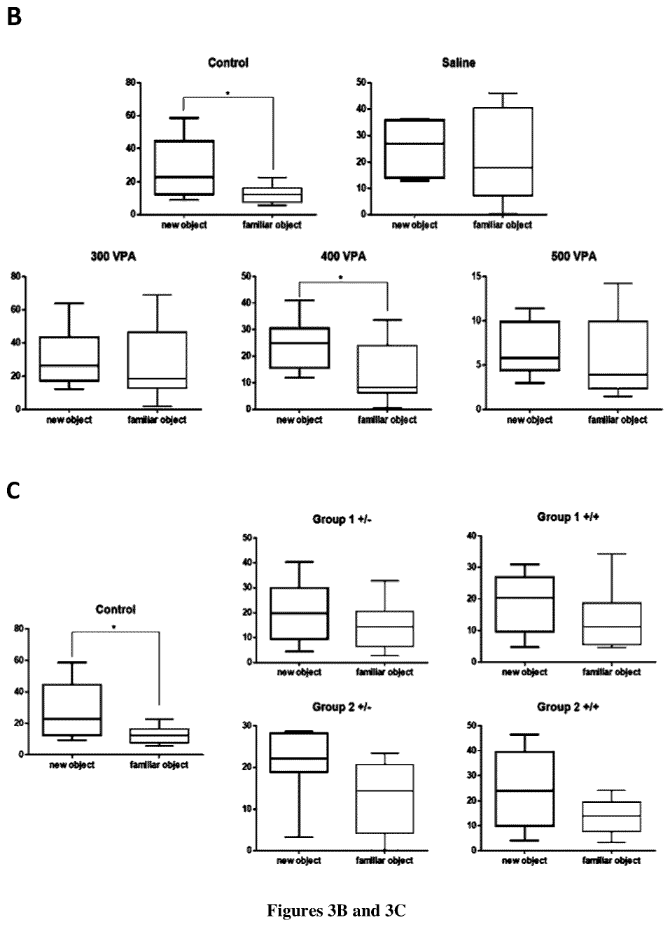

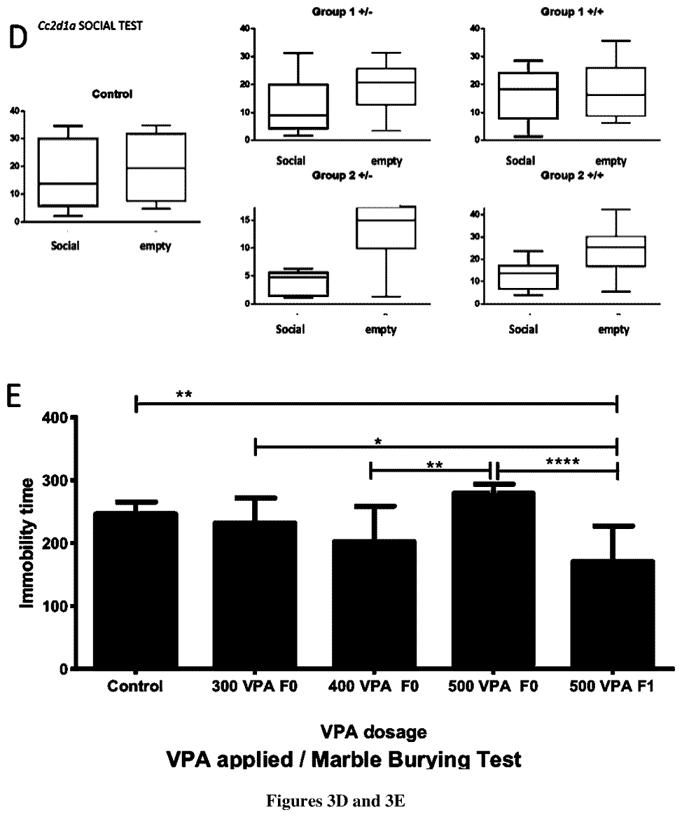

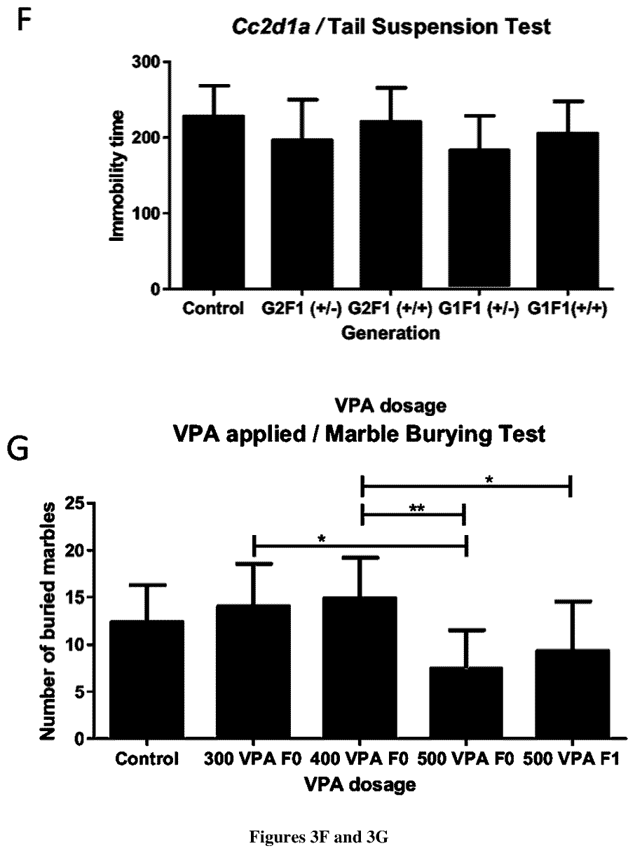

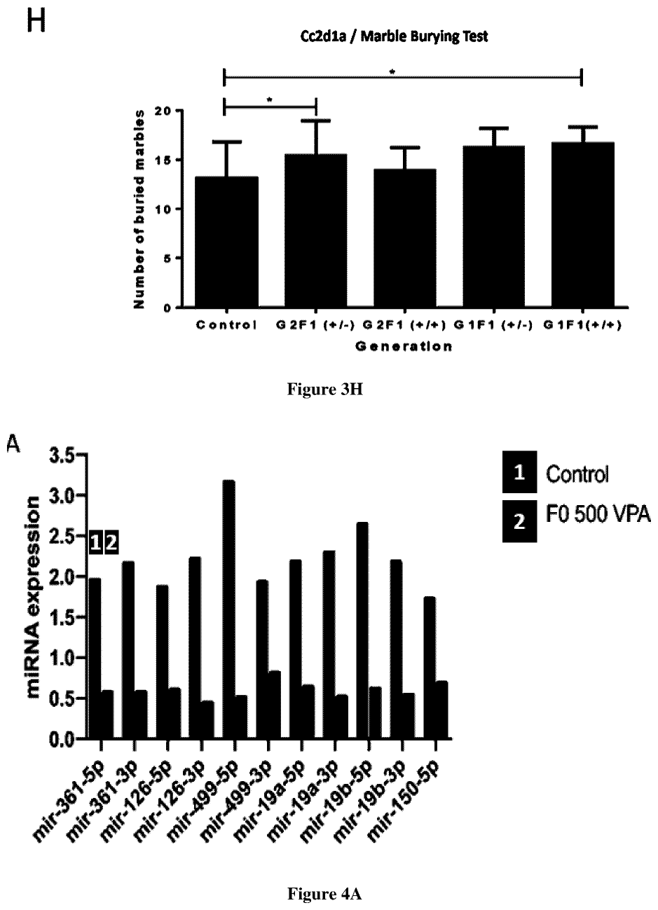

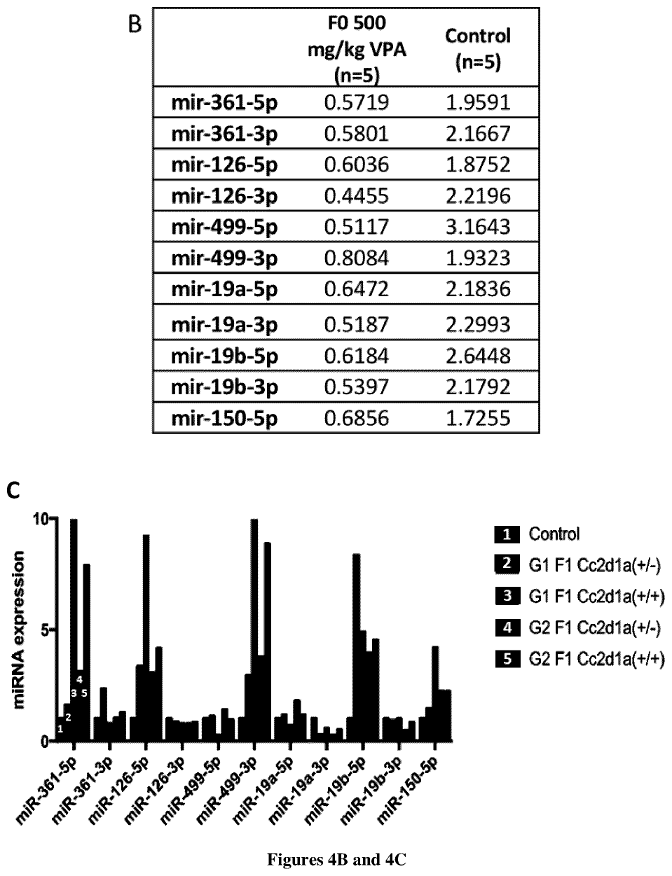

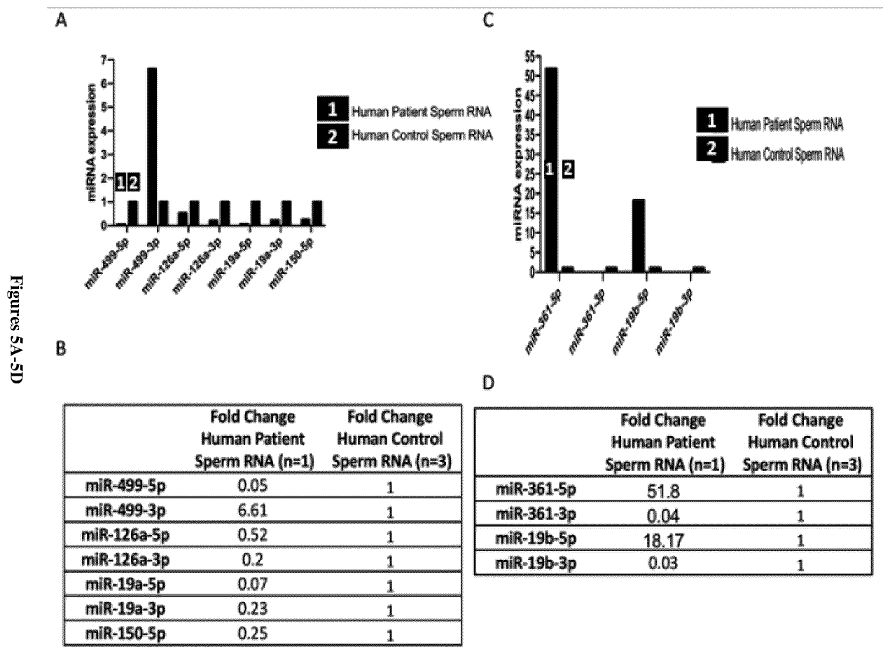

While the number of patients, relatives and controls included in the analysis ensured the statistical validity of the results (p<0.05), their general significance could be questioned because of the apparent divergence of the conclusions from published data generated in much larger cohorts of patients²¹ and the possibility of geographic and ethnographic differences. As a test of the validity of the conclusions regarding the autistic pathology, we checked whether they could be verified in animal models. For this purpose, we chose two distinct models are presented in FIG. 3: mice treated with valproic acid (VPA) and Cc2d1a heterozygotes.

(1) Valproic Acid Intraperitoneal Injection.

VPA is known to perturb brain formation during early development²² and to induce characteristic traits of ASD pathology^{23,24}. To establish reference phenotypes to which the experimental results could be compared, we chose to follow the development of the ASD-like phenotype in B6D2 and Balb c males that had received one intraperitoneal injection of VPA at a concentration of 300-700 mg/kg at two weeks of age²⁵ (data not shown). We treated the animals after birth rather than treating pregnant females as in previous studies^{23,24} to avoid the multiple effects on embryonic development generated via the mothers during pregnancy as well as the effects on germ cells. In addition, we observed that in two-week-old males, VPA injection did not induce changes in body weight compared to the control group, and sudden dramatic mortality only occurred at high doses (600-700 mg/kg) in both mouse line. In present study on the males, doses responses show reproducible affected phenotype at 500 mg/kg dosage without sudden mortality. In contrast, the injection of pregnant females often leads to an unpredicted arrest of embryonic development and mortality of the mothers either during pregnancy or after the birth of their progenies.

Two-week-old mice have not yet completed brain development, and their hippocampal and cerebellar granule cells continue to migrate and differentiate^{26,27-28} and VPA-treated males will develop the characteristic alterations in behavior. The spermatogonial stem cells that will continuously divide and differentiate throughout the life of the male are present, leaving open the possibility that some changes in transcript levels may be transmitted to the next generation. While concentrations of 600 mg/kg and higher were found to be lethal in both genetic backgrounds (data not shown), lower concentrations did not affect body weight or cause any visible phenotype in the mice, such as the previously reported “crooked tail” phenotype²⁴. After the injection of 500 mg/kg VPA, a few males became aggressive toward females during breeding, showed reduced fertility and smaller testes, and were not studied further. The 500 mg/kg dose of valproic acid was reproducibly found optimal for both lines of mice. The exhibited homogenous phenotypes, including characteristic behavior changes and miRNA transcript analysis results are presented only for the Balb c line in FIGS. 3 and 4 of controls and VPA treated 500 mg/kg.

(2) Cc2d1a^{+/-} Heterozygous Mice.

Mice carrying a Cc2d1a knock-out mutation have been reported to present embryonic brain impairment resemble to ASD characteristics²⁹. We purchased mice heterozygous for this mutation (Cc2d1a^{+/-} mice) from the Jackson Laboratory and outbred them into the Balb c background for 10 generations before the initiation of the experimental procedures. Homozygotes (−/−) show lethality beginning at 14 dpc and extending to different embryonic developmental stages in the Balb c line, and no living homozygotes are observed. Heterozygotes (+/−) were established and maintained, as verified by PCR genotyping, according to the instructions of Jackson Laboratory (see Methods and Data not shown for PCR genotyping). Cc2d1a^{+/-} males were mated with normal (+/+) or heterozygous (+/−) females, successively generating G1 and G2 generations.

Further analysis of both models was conducted on groups of two-month-old males (10 males mice) from all groups (data not shown), including Balb c males controls (controls are laboratory mice never been crossed with Cc2d1a^{+/-} mutants mice) subjected to behavioral tests including recognition of a novel versus a familiar object, social interactions, tail suspension and marble burying (see FIG. 3 for the experimental timeline). All analyses were performed in a blinded manner. The results of the behavioral tests and the statistical significance reported in FIG. 3 clearly show the development of altered behavior in the mouse models compared to the control groups that could be related to ASD-like characteristics. The behavioral tests were performed as described in the Methods sections. The same approach could be extended in the future to the others ASD related gene candidates in mouse models.

Behavioral Observations.^{15-17,30,31}

Novel Object Test³²

Object recognition was measured as shown in FIGS. 3A and 3B, according to the difference in the time spent with familiar versus new objects (see Materials and Methods). In fact, all control groups (untreated or treated with peritoneally injected saline) showed more interest in novel objects than familiar objects. The VPA-treated (500 mg/kg) males used as positive controls lost interest and spent the same amount of time with the two objects but spent less time overall with both objects; i.e., there was little distinction between the two objects. Some effect was detectable at a dose of 400 mg/kg, but only for familiar objects. Thus, the effect of VPA was dependent on the dose. In addition, there was a significant difference between the control and VPA groups in the time spent with familiar and new objects and the number of visits (FIG. 3A). A decrease in the overall activity as well by half was also observable in the open field and Morris water tests in the 500 mg/kg VPA group (not shown). Two types of genotypes heterozygous and wild type (living mice) are produced under crossing protocol of G1 (Cc2d1a^{+/-} males were mated with normal (+/+) females) and G2 (Cc2d1a^{+/-} males were mated with Cc2d1a^{+/-} females). From both group (G1 and G2) all genotypes are separately analyzed and here the males with wild type genotype are slightly different from male controls group (males never crossed with Cc2d1a^{+/-} genotype). In the Cc2d1a^{+/-} group (FIG. 3D), the effect was slight but still observable. In fact, slightly less interest in the novel object was observed in the test group compared to the controls, especially in the G1 group. However, some minor differences in examination behavior were observed between the model and controls groups.

Social Interaction Test^{30,33}

The three-chamber social interaction test was originally adapted from Crawley's group and was slightly simplified (see Materials and Methods). Social interaction was measured as shown in FIGS. 3C and 3D, according to the difference in the time spent close to a chamber containing one living mouse compared to an empty chamber. In fact, almost all of the control groups (untreated or treated with peritoneal saline injection) showed no difference in the interaction between the two chambers. The VPA-treated (500 mg/kg) males, used as a positive control, generally showed less interest and spent an even smaller amount of time close to the empty cage (FIG. 3C). Again, the 500 mg/kg VPA results showed marked differences. We took into account all tests involving 500 mg/kg dose-induced phenotypes. A decrease in overall activity in the 500 mg/kg VPA group was also detectable in social interaction tests. In contrast, in the Cc2d1a^{+/-} group (FIG. 3D), we observed a marked difference in social interaction in which more time was spent close to the empty cage rather than the cage with a living mouse. The same effect was observed in the G1+/− and +/+ and G2+/− groups. In contrast, no difference was detected in the G2+/+ test group compared to the controls. The Cc2d1a^{+/-} group did not behave similarly to the VPA group in the social interaction tests, but variation from the control group (never crossed to Cc2d1a^{+/-} genotype) confirmed behavioral changes. The wide variation in ASD phenotypes observed herein in the two ASD-like mouse models compared with the controls is also relevant.

Tail Suspension Test^{30,34}.

In this simple test, a mouse was suspended by its tail with tape, and the movements of the animals in the air were recorded (see Materials and Methods). The total duration was six minutes, and struggle-related behaviors were assessed. Periods of agitation and immobility were recorded. At the 500 mg/kg dose of VPA, a significant change that indicated abandonment and depression was observed (FIG. 3E). In these tests, we observed that the effect was significantly different from what was observed in the controls, but the reverse effect was observed in the F1 VPA group compared to the F0 VPA generation. In the G1 and G2 Cc2d1a^{+/-} groups (FIG. 3F), slight variation from the control was observed but in an opposite direction from the VPA 500 mg/kg behavior. Non-significant differences in tail suspension were observed in the Cc2d1a^{+/-} group.

Marble Burying (MB) Test³⁵

Repetitive action was evaluated in the marble burying test. Twenty glass marbles were placed on the surface of clean bedding, and the number of marbles buried in 30 minutes was scored as described in the Materials and Methods. VPA-treated (500 mg/kg) males (FIG. 3G), used as positive controls, again generally showed markedly less interest in burying marbles. In the 500 mg/kg VPA F1 generation, we observed the same behavior as in the founders, with significantly less activity. In contrast, in the Cc2d1a^{+/-} group (FIG. 3H), we observed slightly more burying activity than in the controls group (never crossed to Cc2d1a^{+/-} genotype).

miRNA Analysis of the Mouse Models.

The six-microRNAs that had been found downregulated in patients were affected to a comparable extent in the two mouse models with altered behavioral traits characteristic of the ASD phenotype FIG. 4 and data not shown. Confirming the association of the observed molecular effects with the disease and paving the way for further investigations not possible in human subjects for obvious practical, ethical and etiological limitations. The low levels of expression of the six-microRNAs downregulated in human sera were paralleled by decreases not only in the blood but also in the sperm and hippocampus of the VPA-treated and Cc2d1a mutant mice group (FIG. 4A-4C and data not shown), with marked differences noted as a subject for future enquiries.

We determined that it was not only the 5p strand sequences but also the 3p (premicroRNA) sequences of the 6 microRNAs that were down-regulated in all cases (FIG. 4A, B and data not shown), The mature and premicroRNA transcripts of a given microRNA share common functional connections, but the downregulation of both strands strongly suggests an alteration in the initial step(s) generating the transcript.

We also show the sperm profiles of the six-miRNAs in the father of three affected children (FIG. 5A-5D).

In the medical and sociological fields, the current conclusions may lead to positive outcomes for the patients and their families. They provide a critical tool to diagnose the disease at the youngest possible age, even at birth, via a simple, non-invasive, inexpensive examination method, thus allowing children to be provided as early as possible with the proper surroundings to facilitate their development.

### REFERENCES

Throughout this application, various references describe the state of the art to which this invention pertains. The disclosures of these references are hereby incorporated by reference into the present disclosure.
1. Iakoucheva, L. M., Muotri, A. R. & Sebat, J. Getting to the Cores of Autism. Cell vol. 178 1287-1298 (2019).
2. Auerbach, B. D., Osterweil, E. K. & Bear, M. F. Mutations causing syndromic autism define an axis of synaptic pathophysiology. Nature 480, 63-68 (2011).
3. Liu, L. et al. DAWN: A framework to identify autism genes and subnetworks using gene expression and genetics. Mol. Autism 5, (2014).
4. Satterstrom, F. K. et al. Large-Scale Exome Sequencing Study Implicates Both Developmental and Functional Changes in the Neurobiology of Autism. Cell (2020) doi:10.1016/j.cell.2019.12.036.
5. Kazdoba, T. M., Leach, P. T. & Crawley, J. N. Behavioral phenotypes of genetic mouse models of autism. Genes, Brain and Behavior (2016) doi:10.1111/gbb.12256.
6. Kleaveland, B., Shi, C. Y., Stefano, J. & Bartel, D. P. A Network of Noncoding Regulatory RNAs Acts in the Mammalian Brain. Cell (2018) doi:10.1016/j.cell.2018.05.022.
7. Bartel, D. P. Metazoan MicroRNAs. Cell (2018) doi:10.1016/j.cell.2018.03.006.
8. Rajman, M. & Schratt, G. MicroRNAs in neural development: From master regulators to fine-tuners. Development (Cambridge) (2017) doi:10.1242/dev.144337.
9. Ghahramani Seno, M. M. et al. Gene and miRNA expression profiles in autism spectrum disorders. Brain Res. (2011) doi:10.1016/j.brainres.2010.09.046.
10. Da Silva Vaccaro, T. et al. Alterations in the microRNA of the blood of autism spectrum disorder patients: Effects on epigenetic regulation and potential biomarkers. Behav. Sci. (Basel). 8, (2018).
11. Ardinger, H. H. et al. Verification of the fetal valproate syndrome phenotype. American Journal of Medical Genetics (1988) doi:10.1002/ajmg.1320290123.
12. Mutlu-Albayrak, H., Bulut, C. & Qaksen, H. Fetal Valproate Syndrome. Pediatr. Neonatol. 58, 158-164 (2017).
13. Christianson, A. L., Chester, N. & Kromberg, J. G. R. Fetal Valproate Syndrome: Clinical and Neuro-developmental Features in Two Sibling Pairs. Dev. Med. Child Neurol. (1994) doi:10.1111/j.1469-8749.1994.tb11858.x.
14. Schneider, T. & Przewlocki, R. Behavioral alterations in rats prenatally to valproic acid: Animal model of autism. Neuropsychopharmacology (2005) doi:10.1038/sj.npp.1300518.
15. Chessa, L. & Tannetti, P. Fetal valproate syndrome. American Journal of Medical Genetics (1986) doi:10.1002/ajmg.1320240221.
16. Albert, P. R., Vahid-Ansari, F. & Luckhart, C. Serotonin-prefrontal cortical circuitry in anxiety and depression phenotypes: Pivotal role of pre- and post-synaptic 5-HT1A receptor expression. Frontiers in Behavioral Neuroscience vol. 8 (2014).
17. Al-Tawashi, A., Jung, S. Y., Liu, D., Su, B. & Qin, J. Protein implicated in nonsyndromic mental retardation regulates protein kinase A (PKA) activity. J. Biol. Chem. (2012) doi:10.1074/jbc.M111.261875.
18. Jacobs, B. L. & Azmitia, E. C. Structure and function of the brain serotonin system.

Physiological Reviews (1992) doi:10.1152/physrev.1992.72.1.165.
19. Nichols, C. D. Serotonin. in Encyclopedia of the Neurological Sciences (2014). doi:10.1016/B978-0-12-385157-4.00048-8.
20. Carr, G. V. & Lucki, I. The role of serotonin receptor subtypes in treating depression: A review of animal studies. Psychopharmacology (2011) doi:10.1007/s00213-010-2097-z.
21. Hicks, S. D. & Middleton, F. A. A comparative review of microRNA expression patterns in autism spectrum disorder. Front. Psychiatry (2016) doi:10.3389/fpsyt.2016.00176.
22. Nau, H., Hauck, R.-S & Ehlers, K. Valproic Acid—Induced Neural Tube Defects in Mouse and Human: Aspects of Chirality, Alternative Drug Development, Pharmacokinetics and Possible Mechanisms. Pharmacol. Toxicol. 69, 310-321 (1991).
23. Wagner, G. C., Reuhl, K. R., Cheh, M., McRae, P. & Halladay, A. K. A new neurobehavioral model of autism in mice: Pre- and postnatal exposure to sodium valproate. J. Autism Dev. Disord. (2006) doi:10.1007/s10803-006-0117-y.
24. Choi, C. S. et al. The transgenerational inheritance of autism-like phenotypes in mice exposed to valproic acid during pregnancy. Sci. Rep. 6, (2016).
25. Mabunga, D. F. N., Gonzales, E. L. T., Kim, J., Kim, K. C. & Shin, C. Y. Exploring the Validity of Valproic Acid Animal Model of Autism. Exp. Neurobiol. (2015) doi:10.5607/en.2015.24.4.285.
26. Ellegood, J. et al. Clustering autism: Using neuroanatomical differences in 26 mouse models to gain insight into the heterogeneity. Mol. Psychiatry (2015) doi:10.1038/mp.2014.98.
27. DiCicco-Bloom, E. et al. The developmental neurobiology of autism spectrum disorder. Journal of Neuroscience vol. 26 6897-6906 (2006).
28. Nagode, D. A. et al. Abnormal Development of the Earliest Cortical Circuits in a Mouse Model of Autism Spectrum Disorder. Cell Rep. (2017) doi:10.1016/j.celrep.2017.01.006.
29. Zhao, M., Raingo, J., Chen, Z. J. & Kavalali, E. T. Cc2d1a, a C2 domain containing protein linked to nonsyndromic mental retardation, controls functional maturation of central synapses. J. Neurophysiol. 105, 1506-1515 (2011).
30. Moy, S. S., Nadler, J. J., Magnuson, T. R. & Crawley, J. N. Mouse models of autism spectrum disorders: The challenge for behavioral genetics. American Journal of Medical Genetics—Seminars in Medical Genetics (2006) doi:10.1002/ajmg.c.30081.
31. Silverman, J. L., Yang, M., Lord, C. & Crawley, J. N. Behavioural phenotyping assays for mouse models of autism. Nature Reviews Neuroscience (2010) doi:10.1038/nrn2851.
32. Grayson, B. et al. Assessment of disease-related cognitive impairments using the novel object recognition (NOR) task in rodents. Behav. Brain Res. (2015) doi:10.1016/j.bbr.2014.10.025.
33. Moy, S. S. & Nadler, J. J. Advances in behavioral genetics: Mouse models of autism. Molecular Psychiatry (2008) doi:10.1038/sj.mp.4002082.
34. Powell, T. R., Fernandes, C. & Schalkwyk, L. C. Depression-Related Behavioral Tests. Curr. Protoc. Mouse Biol. (2012) doi:10.1002/9780470942390.mo110176.
35. Albelda, N. & Joel, D. Animal models of obsessive-compulsive disorder: Exploring pharmacology and neural substrates. Neuroscience and Biobehavioral Reviews (2012) doi:10.1016/j.neubiorev.2011.04.006.
36. Hu, Y., Ehli, E. A. & Boomsma, D. I. MicroRNAs as biomarkers for psychiatric disorders with a focus on autism spectrum disorder: Current progress in genetic association studies, expression profiling, and translational research. Autism Research vol. 10 1184-1203 (2017).
37. Chu, M. et al. MicroRNA-126 participates in lipid metabolism in mammary epithelial cells. Mol. Cell. Endocrinol. 454, 77-86 (2017).
38. Kangas, R. et al. Aging and serum exomiR content in women-effects of estrogenic hormone replacement therapy. Sci. Rep. (2017) doi:10.1038/srep42702.
39. Rassoulzadegan, M. et al. RNA-mediated non-mendelian inheritance of an epigenetic change in the mouse. Nature 441, (2006).
40. Gapp, K. et al. Implication of sperm RNAs in transgenerational inheritance of the effects of early trauma in mice. Nat. Neurosci. (2014) doi:10.1038/nn.3695.
41. Livak, K. J. & Schmittgen, T. D. Analysis of relative gene expression data using real-time quantitative PCR and the 2-ΔΔCT method. Methods 25, 402-408 (2001).
42. Analysis of Relative Gene Expression Data Using Real-Time Quantitative PCR and the 2(−Delta Delta C(T)) Method—PubMed. https://pubmed.ncbi.nlm.nih.gov/11846609-analysis-of-relative-gene-expression-data-using-real-time-quantitative-pcr-and-the-2-delta-delta-ct-method/?from_term=Livak+2001.

1. A method for diagnosing autism spectrum disorder (ASD) in a subject comprising the steps of: i) determining the level of at least one of miR-19a-3p, miR-126-3p, miR-499a-5p, miR-361-5p, miR-3613-3p and/or miR-150-5p in a biological sample from the subject ii) determining that the level of at least one of miR-19a-3p, miR-126-3p, miR-499a-5p, miR-361-5p, miR-3613-3p and/or miR-150-5p is lower than a corresponding predetermined reference value and iii) administering to the subject determined to have a low level of at least one of miR-19a-3p, miR-126-3p, miR-499a-5p, miR-361-5p, miR-3613-3p and/or miR-150-5p a therapeutically effective amount of a compound and/or condition which increases the expression level of miR-19a-3p, miR-361-5p, miR-3613-3p, miR-150-5p, miR-126-3p, and/or miR-499a-5p.

2. The method according to claim 1 wherein the subject is at risk to give birth to ASD children.

3. The method according to claim 1, wherein, the biological sample is blood, saliva, breast milk, urine, semen, blood plasma, synovial fluid, hippocampus or serum.

4. The method according to claim 1, wherein the level miR-19a-3p, miR-361-5p, miR-3613-3p, miR-150-5p, miR-126-3p, and miR-499a-5p is determined by quantitative Real-Time Polymerase Chain Reaction (qRT-PCR).

5. A method of treating ASD in a subject in need thereof comprising administering to the subject a therapeutically effective amount of a compound and/or condition which increases the expression level of miR-19a-3p, miR-361-5p, miR-3613-3p, miR-150-5p, miR-126-3p, and/or miR-499a-5p.

6. The method according to claim 5 wherein the subject is determined to suffer from ASD by determining that the level of miR-19a-3p, miR-361-5p, miR-3613-3p, miR-150-5p, miR-126-3p, and/or miR-499a-5p in a biological sample from the subject is lower than a corresponding predetermined reference value.

7. The method according to claim 5 wherein said compound is an agomir.

8. The method according to claim 5 wherein said compound is Clozapine.

9. The method according to claim 5 wherein said compound is selected from the group consisting of: antipsychotics; antidepressants; stimulants; anticonvulsants; Revia; Xanax; Effexor; and Anafranil.

10. A pharmaceutical composition comprising a compound which increases the expression level of miR-19a-3p, miR-361-5p, miR-3613-3p, miR-150-5p, miR-126-3p, and/or miR-499a-5p.11.

11. (canceled)

12. (canceled)

13. A kit for performing the method according to claim 1, wherein said kit comprises (i) means for determining the level of the miR-19a-3p, miR-361-5p, miR-3613-3p, miR-150-5p, miR-126-3p, and/or miR-499a-5p in a biological sample obtained from a subject who is suffering or is susceptible to suffer from ASD and (ii) instructions for comparing with a reference value.
